SEQUENCE LISTING (48 sequences)

SEQ ID NO: 1 - DNA, 504 bp, Ophiophagus hannah
  misc_feature (350)..(350): n is a or g or c or t
gacactacactttgcagtctccagagaagatcgcgagatgaaaactctgctgctgaccttggtggtggtgacaatcgtgtgcctggacttaggatacaccaggaaatgtctcaacacaccgcttcctttgatctatacgacttgtccaatagggcaggacaaatgcgttaaaatgactattaagaagttgccatctaaatatgacgtcatcaggggatgtattgatatttgccctaaaagcagtgccgatgtggaagttctgtgttgcgatacaaacaaatgcaacaaatagctctacgagtggctaaattccttgagttttgctctcatccatcatggaccatccttgnaaaatttatgcttgtggcctataccaccagatggtccatcatccccctctcccctgcctttctttgactcctcatcatctttcccttttctcttgttctgtatgtttccttctgctagttctgtagtttgagaatcaaataaacctcagcattc

SEQ ID NO: 2 - protein, 84 aa, Ophiophagus hannah
MKTLLLTLVVVTIVCLDLGYTRKCLNTPLPLIYTTCPIGQDKCVKMTIKKLPSKYDVIRGCIDICPKSSADVEVLCCDTNKCNK

SEQ ID NO: 3 - protein, 63 aa, Ophiophagus hannah
RKCLNTPLPLIYTTCPIGQDKCVKMTIKKLPSKYDVIRGCIDICPKSSADVEVLCCDTNKCNK

SEQ ID NO: 4 - DNA, 475 bp, Ophiophagus hannah
atgaaaactctgctgctgaccttggtggtggtgacaatcgtgtgcctggacttaggatacaccaggaaatgtctcaacacaccgcttcctttgatctataagacttgtccaatagggcaggacaaatgcattaaaatgactattaagaagttgccatctaaatatgacgtcatcaggggatgtattgatatttgccctaaaagcagtgccgatgtggaagttctgtgttgcgatacaaacaaatgcaacaaatagctctacgagtggctaaattccttgagttttgctctcaaccatcatggaccatccttgaaaatttatgcttgtggcctataccaccagatggtccatcatccccctctcccctgctttctttgactcctcatcatctttcccttttctctcgttctgtaagtttccttctgctagttctgtagtttgagaatcaaataaacctcagcaaaaaaaaaaaaaa

SEQ ID NO: 5 - protein, 84 aa, Ophiophagus hannah
MKTLLLTLVVVTIVCLDLGYTRKCLNTPLPLIYKTCPIGQDKCIKMTIKKLPSKYDVIRGCIDICPKSSADVEVLCCDTNKCNK

SEQ ID NO: 6 - protein, 63 aa, Ophiophagus hannah
RKCLNTPLPLIYKTCPIGQDKCIKMTIKKLPSKYDVIRGCIDICPKSSADVEVLCCDTNKCNK

SEQ ID NO: 7 - DNA, 479 bp, Ophiophagus hannah
atgaaaactctgctgctgaccttggtggtggtgacaatcgtgtgcctggacttaggatacaccaggaaatgtctcaacacaccgcttcctttgatctataagacttgtccaatagggcaggacagatgcattaaaatgactattaagaagttgccatctaaatatgacgtcatcaggggatgtattgatatttgccctaaaagcagtgccgatgtggaagttctgtgttgcgatacaaacaaatgcaacaaatagctctacgagtggctaaattccttgagttttgctctcaaccatcatggaccatccttgaaaatttatgcttgtggcctataccaccagatggtccatcatccccctctcccctgctttctttgactcctcatcatctttcccttttctctcgttctgtaagtttccttctgctagttctgtagtttgagaatcaaataaacctcagcatccaaaaaaaaaaaaaa

SEQ ID NO: 8 - protein, 84 aa, Ophiophagus hannah
MKTLLLTLVVVTIVCLDLGYTRKCLNTPLPLIYKTCPIGQDRCIKMTIKKLPSKYDVIRGCIDICPKSSADVEVLCCDTNKCNK

SEQ ID NO: 9 - protein, 63 aa, Ophiophagus hannah
RKCLNTPLPLIYKTCPIGQDRCIKMTIKKLPSKYDVIRGCIDICPKSSADVEVLCCDTNKCNK

SEQ ID NO: 10 - DNA, 498 bp, Ophiophagus hannah
atgaaaactctgctgctgaccttggtggtggtgacaatcgtgtgcctggacttaggatacaccaggaaatgtctcaacacaccgcttcctttgatctataagacttgtccaatagggcaggacaaatgcattaaaatgactattaagaagttgccatctaaatatgacgttatcaggggatgtactgatatttgccctaaaagcagtgccgatgtggttgttgtgtgttgcgatacaaacaaatgcaacaaatagctctatgagtggctaaattccttgagttttgctctcaaccatcatggaccatccttgaaaatttatgcttgtggcctataccaccagatggtccatcatccccctctcccctgctttctttgactcctcatcatctttcccttttctcttgttctgtaagtttccttctgctagttctgtagtttgagaatcaaataaacctcagcattcaattcaaaaaaaaaaagtaaaaaaaaaaaaaaa

SEQ ID NO: 11 - protein, 84 aa, Ophiophagus hannah
MKTLLLTLVVVTIVCLDLGYTRKCLNTPLPLIYKTCPIGQDKCIKMTIKKLPSKYDVIRGCTDICPKSSADVVVVCCDTNKCNK

SEQ ID NO: 12 - protein, 63 aa, Ophiophagus hannah
RKCLNTPLPLIYKTCPIGQDKCIKMTIKKLPSKYDVIRGCTDICPKSSADVVVVCCDTNKCNK

SEQ ID NO: 13 - DNA, 480 bp, Ophiophagus hannah
atgaaaactctgctgctgaccttggtggtggtgacaatcgtgtgcctggacttaggatacaccaggaaatgtctcaacacaccgcttcctttgatctatacgacttgtccaatagggcaggacaaatgcgttaaaatgactattaagaagttgccatctaaatatgacgttatcaggggatgtactgatatttgccctaaaagcagtgccgatgtggttgttgtgtgttgcgatacaaacaaatgcgacaaatagctctatgagtggctaaattccttgagttttgctctcaaccatcatggaccatccttgaaaatttatgcttgtggcctataccaccagatggtccatcatccccctctcccctgctgtctttgacacctcattgcctttcccttttctcttgttctgtaagtttccttctgctagttctgtagtttgagaatcaaataaacctcagcattgaaaaaaaaaaaaaaa

SEQ ID NO: 14 - protein, 84 aa, Ophiophagus hannah
MKTLLLTLVVVTIVCLDLGYTRKCLNTPLPLIYTTCPIGQDKCVKMTIKKLPSKYDVIRGCTDICPKSSADVVVVCCDTNKCDK

SEQ ID NO: 15 - protein, 63 aa, Ophiophagus hannah
RKCLNTPLPLIYTTCPIGQDKCVKMTIKKLPSKYDVIRGCTDICPKSSADVVVVCCDTNKCDK

SEQ ID NO: 16 - DNA, 504 bp, Ophiophagus hannah
atgaaaactctgctgctgaccttggtggtggtgacaatcgtgtgcctggacttaggatacaccaggaaatgtctcaacacaccgcttcctttgatctatacgacttgtccaatagggcaggacaaatgcgttaaaatgactattaagaagttgccatctaaatatgacgttatcaggggatgtactgatatttgccctaaaagcagtgccgatgtggttgttgtgtgttgcgatacaaacaaatgcaacaaatagctctatgagtggctaaattccttgagttttgctctcaaccatcatggaccatccttgaaaatttatgcttgtggcctataccaccagatggtccatcatccccctctcccctgctgtctttgacacctcattgcctttcccttttctcttgttctgtaagtttccttctgctagttctgtagtttgagaatcaaataaacctcagcattcaaaaaaaaaaaaaaaaaaaaaacaaaaaaaaaaaaaaaa

SEQ ID NO: 17 - protein, 84 aa, Ophiophagus hannah
MKTLLLTLVVVTIVCLDLGYTRKCLNTPLPLIYTTCPIGQDKCVKMTIKKLPSKYDVIRGCTDICPKSSADVVVVCCDTNKCNK

SEQ ID NO: 18 - protein, 63 aa, Ophiophagus hannah
RKCLNTPLPLIYTTCPIGQDKCVKMTIKKLPSKYDVIRGCTDICPKSSADVVVVCCDTNKCNK

SEQ ID NO: 19 - protein, 126 aa, Artificial sequence
  Synthetic sequence
RKCLNTPLPLIYXXXXXXXXXXTCPIGQDXXXXXXXXXXCXXXXXXXXXXKMTIKKLPSKYDVIRGCXXXXXXXXXXDICPKSSADVXXXXXXXXXXVXXXXXXXXXXCCDTNKCXXXXXXXXXXK

SEQ ID NO: 20 - protein, 12 aa, Ophiophagus hannah
RKCLNTPLPLIY

SEQ ID NO: 21 - protein, 7 aa, Ophiophagus hannah
TCPIGQD

SEQ ID NO: 22 - protein, 21 aa, Artificial sequence
  Synthetic sequence
XXXXXXXXXXCXXXXXXXXXX

SEQ ID NO: 23 - protein, 17 aa, Ophiophagus hannah
KMTIKKLPSKYDVIRGC

SEQ ID NO: 24 - protein, 10 aa, Ophiophagus hannah
DICPKSSADV

SEQ ID NO: 25 - protein, 21 aa, Artificial sequence
  Synthetic sequence
XXXXXXXXXXVXXXXXXXXXX

SEQ ID NO: 26 - protein, 18 aa, Artificial sequence
  Synthetic sequence
CCDTNKCXXXXXXXXXXK

SEQ ID NO: 27 - protein, 60 aa, Naja nivea
LKCHKLVPPVWKTCPEGKNLCYKMFMVSTSTVPVKRGCIDVCPKDSALVKYVCCSTDKCN

SEQ ID NO: 28 - protein, 60 aa, Naja haje
LKCHKLVPPVWKTCPEGKNLCYKMFMVSTSTVPVKRGCIDVCPKNSALVKYVCCSTDKCN

SEQ ID NO: 29 - protein, 60 aa, Naja annulifera
LECNKLVPIAHKTCPEGKNLCYKMFMVSTSTVPVKRGCIDVCPKDSALVKYVCCNTDRCN

SEQ ID NO: 30 - protein, 60 aa, Naja nivea
LKCHQLIPPFWKTCPEGKNLCYKMYMVATPMIPVKRGCIDVCPKNSALVKYMCCNTDKCN

SEQ ID NO: 31 - protein, 62 aa, Ophiophagus hannah
RICLKQEPFQPETTTTCPEGEDACYNLFWSDHSEIKIEMGCGCPKTEPYTNLYCCKIDSCNK

SEQ ID NO: 32 - protein, 62 aa, Ophiophagus hannah
LICFNQETYRPETTTTCPDGEDTCYSTFWNDHHGVKIERGCGCPRVNPPISIICCKTDKCNN

SEQ ID NO: 33 - protein, 62 aa, Ophiophagus hannah
LICFNQETYRPETTTTCPDGEDTCYSTFWNDHRGVKIERGCGCPRVNPGISIICCKTDKCNN

SEQ ID NO: 34 - protein, 60 aa, Ophiophagus hannah
LICFNQETYRPETTTTCPDGENCYSTFWHNDGHVKIERGCGCPRVNPPISICCKTDKCNN

SEQ ID NO: 35 - protein, 62 aa, Laticauda colubrine
RRCFNQQSSQPKTTKSCPLGENSCYNKQWRDHRGSITERGCGCPKVKPGIKLRCCESEDCNN

SEQ ID NO: 36 - protein, 65 aa, Ophiophagus hannah
TKCYNHQSTTPETTEICPDSGYFCYKSSWIDGREGRIERGCTFTCPELTPNGKYVYCCRRDKCNQ

SEQ ID NO: 37 - protein, 65 aa, Bungarus multicinctus
TICYNHLSRTPETTEICPDSWYFCYKISLADGNDVRIKRGCTFTCPELRPTGKYVYCCRRDKCNQ

SEQ ID NO: 38 - protein, 65 aa, Naja kaouthia
TICYNHLTRTSETTEICPDSWYFCYKISLADGNDVRIKRGCTFTCPELRPTGIYVYCCRRDKCNQ

SEQ ID NO: 39 - protein, 65 aa, Bungarus multicinctus
KTCFNDDLTNPKTTELCRHSMYFCFKNSWIAGGVERIERGCSLTCPDIKYNGKYIYCCTRDNCNA

SEQ ID NO: 40 - protein, 65 aa, Bungarus candidus
KTCFNDNLANPKTTELCRHSMYFCFKNSWIAGGVERIERGCSLTCPDIKYNGKYIYCCTRDNCNA

SEQ ID NO: 41 - protein, 72 aa, Ophiophagus hannah
TKCYVTPDATSQTCPDGENICYTKSWCDVFCSSRGKRIDLGCAATCPKVKPGVDIKCCSTDNCNPFTPWKRH

SEQ ID NO: 42 - protein, 72 aa, Ophiophagus hannah
TKCYVTPDVKSETCPDGENICYTKSWCEVFCTSRGKRIDLGRAATCPKVKPGVDIKCCSTDNCNPFTPWKRH

SEQ ID NO: 43 - protein, 72 aa, Ophiophagus hannah
TKCYITPDVKSETCPDGENICYTKSWCDVFCTSRGKRIDLGCAATCPKVKPGVDIKCCSTDNCNPFTPWKRH

SEQ ID NO: 44 - protein, 73 aa, Ophiophagus hannah
TKCYVTPDATSQTCPDGQDICYTKTWCDGFCSSRGKRIDLGCAATCPKVKPGVDIKCCSTDNCNPFPTWKRKH

SEQ ID NO: 45 - protein, 70 aa, Ophiophagus hannah
TKCYVTPDVTSQTCPDGQNICYTETWCDAWCGSRGKRVNLGCAATCPKVNPGVDIICCSTDNCNPFPKRS

SEQ ID NO: 46 - protein, 10 aa, Artificial sequence
  Synthetic sequence BCL1a
LPLIYTTAPI

SEQ ID NO: 47 - protein, 12 aa, Artificial sequence
  Synthetic sequence BCL1b
TPLPLIYTTAPI

SEQ ID NO: 48 - protein, 21 aa, Artificial sequence
  Synthetic sequence BCL2
KCVKMTIKKLPSKYDVIRGCI

(19) United States
(12) Patent Application Publication
(10) Pub. No.: US 2010/0062980 A1
(43) Pub. Date: Mar. 11, 2010

(54) THERAPEUTIC USES OF BETA-ANTAGONISTS

(75) Inventors: Manjunatha Ramachandra Kini, Singapore (SG); Yuh Fen Pung, Singapore (SG); Yi Zhun Zhu, Singapore (SG); Tsun Hon Peter Wong, Singapore (SG); Prakash Kumar, Singapore (SG); Nandhakishore Rajagopalan, Singapore (SG)

Correspondence Address: SPECKMAN LAW GROUP PLLC, 1201 THIRD AVENUE, SUITE 330, SEATTLE, WA, 98101, US

(73) Assignee: NATIONAL UNIVERSITY OF SINGAPORE, Singapore (SG)

(21) Appl. No.: 12/516,550

(22) PCT Filed: Nov. 26, 2007
(86) PCT No.: PCT/SG2007/000405

§ 371 Date: May 27, 2009

Related U.S. Application Data
(60) Provisional application No. 60/867,301, filed on Nov. 27, 2006.

(51) Int. Cl. A61K 38/16; C07K 14/00; A61P 9/00

(52) U.S. Cl. 514/12; 530/324

(57) ABSTRACT

Polypeptides derived from Ophiophagus hannah (king cobra) are disclosed together with their use in the treatment of disease, disorder or pathological condition in a patient in need of treatment. The polypeptides disclosed are functionally characterised by their ability to reduce the mammalian heart rate and/or their β-antagonist activity.

### FIELD OF THE INVENTION

The present invention relates to therapeutic use of polypeptides and particularly, although not exclusively, to polypeptides that exhibit β-antagonist activity and/or are capable of reducing the heart rate of a mammal.

### BACKGROUND TO THE INVENTION

Snake venoms have been a cornucopia of bioactive proteins and polypeptides (1, 2). This arsenal of pharmacologically active molecules have been utilized in the past for obtaining several therapeutic agents and lead compounds like bradykinin-potentiating peptides for the inhibition of angiotensin converting enzyme (3), eptifibatide and tirofiban for the inhibition of platelet aggregation (4-9) and ancrod for the reduction of blood fibrinogen levels (10). Snake venom toxins have also found extensive application as research tools. For example, α-bungarotoxin has been used to characterize the fundamental mechanisms involved in neuromuscular transmission (11).

Proteins from snake venoms fall under two categories, namely enzymatic and non-enzymatic proteins. Among non-enzymatic proteins, three-finger toxin (3FTX) family is the most abundant and well-characterized family. All members share a similar fold consisting of three finger-like loops made of β-sheet structure, projecting from a globular core and stabilized by 4 or 5 intra-molecular disulfide linkages giving rise to the compact ‘β-cross’ motif (12-16). Despite the similar structural fold different 3FTXs have diverse molecular targets in the prey. For example, short and long-chain α-neurotoxins target α1 nicotinic acetylcholine receptors (nAChRs), long-chain α-neurotoxins target α7 nAChR, κ-bungarotoxins target α3 and α4 nAChRs (15), muscarinic toxins target muscarinic AchRs (17), fasciculin targets acetylcholinesterase (18), calciseptine and FS2 toxin target L-type calcium channel (19), dendroaspin targets integrin α_{IIb}β₃ (20), cardiotoxins target phospholipids and glycosphingolipids (21), hemextin AB complex targets coagulation factor VIIa (22), and cardiotoxin A5 targets integrin αᵥβ₃ (23). However, there are a number of ‘orphan groups’ of 3FTXs whose molecular target in the prey and hence their functional roles in the snake venom are not yet known (24).

There have been reports of cytotoxin-like proteins purified from Ophiophagus hannah (king cobra) (Chang et al Toxicon 2006 Sep. 15; 48(4): 429-36. Epub 2006 Jun. 29; and Li et al Biochem J. 2006 Sep. 1; 398 (2):233-42). However, the biological properties of these proteins have not been characterized.

Three distinct types of β-adrenergic receptors (β-adrenoreceptors; β-AR) have been clearly identified β₁, β₂ and β₃) and there is evidence for a fourth type (β₄). β₁-adrenergic receptors are located primarily in cardiac tissue, and β₂-adrenergic receptors are located in smooth muscle and other tissue. β-antagonists are known to be employed as antihypertensives and for treating cardiac arrhythmias and cardiac failure. Known β-antagonists are small molecules, and most of the clinically relevant β-antagonists (β-blockers) are from the chemical class of aryloxypropanolamines, such a propranolol.

Though widely used, physicians have encountered several problems associated with the currently available beta-blockers. All beta-blockers are available as racemic mixtures of L- and D-enantiomers, where only the L-enantiomer exerts beta-blockade while the D-enantiomer apart from being inert may have adverse side effects. For example, in a clinical trial using the D-enantiomer of the widely used beta-blocker sotalol, it was reported that the mortality increased by 65% compared to placebo. Many beta-blockers exhibit varying levels of lipophilicity and so have the ability to cross membrane barriers and reach the central nervous system, causing adverse effects like hallucinations and insomnia. Some beta-blockers were shown to increase insulin resistance and raise the risk of diabetes. Thus, new beta-blockers with high specificity and low side effects are being sought.

A review of the adrenergic receptor family and antagonist agents is provided in Adrenergics and Adrenergic Blocking Agents, Robert K. Griffith, Burger's Medicinal. Chemistry and Drug Discovery, Sixth Edition, Volume 6: Nervous System Agents, Edited by Donald J. Abraham (2003 John Wiley & Sons, Inc).

Cardiovascular diseases (CVD) are widespread and are a major health issue in the developed nations. For example, a recent study revealed that about 71.3 million American adults suffer from one or more forms of CVD. CVD causes nearly every 1 in 3 deaths and was the number one killer as it was the cause for 37.3% deaths in the year 2003. The economic impact of CVD is huge, and it is estimated that the direct and indirect costs of treating CVDs in 2006 would be $403.1 billion. Beta-blockers, which are antagonists of β-adrenergic receptors (ARs), that are expressed in cardiomyocytes are the drugs of choice in the treatment of CVDs. 93% of patients with myocardial infarction are given beta-blockers on arrival at hospitals. They are also prescribed after discharge as a chronic therapy. In addition, beta-blockers are used in the treatment of many pathological conditions afflicting the heart and vasculature, such as ventricular arrhythmias, heart failure, digitalis intoxication and fetal tachycardia.

Owing to the presence of β-ARs in various other tissues, beta-blockers have also been used to treat conditions like, migraine, essential tremor, situational anxiety, alcohol withdrawal, hyperparathyroidism, glaucoma, portal hypertension and gastrointestinal bleeding.

Pullar and co-workers recently demonstrated that β₂-AR activation leads to a delay in skin wound healing. They went on to exploit this finding and showed, by using non-specific beta-blockers, that β₂-AR blockade accelerated the wound healing process.

### SUMMARY OF THE INVENTION

Snake venoms have provided a number of novel ligands with therapeutic potential. The inventors have constructed a partial cDNA library from the mRNA of Ophiophagus hannah (king cobra) venom gland tissue and identified five genes encoding proteins belonging to the three-finger toxin family of snake venom proteins. The inventors have isolated and functionally characterized a protein, named β-cardiotoxin (SEQ ID No.3), from the crude venom. It is a small, β-sheet containing protein with a molecular weight of 7012.43±0.91 Da. It has a unique ‘molten globule’ state with an unusual α-helical conformation in the thermal unfolding pathway of the protein.

The protein was found to be not lethal up to a dose of 10 mg/kg when injected through the intraperitoneal route into mice. It induces labored breathing and death at a dose of 100 mg/kg. It does not show any hemolytic or anticoagulant activity in vitro. It caused a dose-dependent decrease of the heart rate in vivo (in anesthetized rat) and also ex vivo (in Langendorff preparations of isolated rat heart). This is in contrast to the classical cardiotoxins from snake venom which increase the heart rate in animals. Radioligand displacement studies showed that this protein targets β-adrenergic receptors with a binding affinity (Kᵢ) of 5.3 μM and 2.3 μM towards β₁ and β₂ subtypes, respectively, to bring about its effect. This is the first report of an exogenous peptide/polypeptide beta-blocker.

The present invention provides polypeptide beta-blockers having antagonist activity towards β₁- and/or β₂-adrenergic receptors together with the nucleotide sequences of nucleic acids encoding such polypeptides which are useful for the expression of the polypeptide beta-blockers. In particular, the present invention provides the use of such polypeptide beta-blockers in therapeutic applications to treat any disease, disorder or pathological condition that may be treated by antagonist action of β- (preferably β₁- and/or β₂-) adrenergic receptors.

In accordance with this invention a polypeptide is provided that has been shown to reduce the mammalian heart rate and, as such, polypeptides of this class are useful in, and are provided for the treatment of all types of disease, disorder or pathological condition, including cardiovascular disease, in which reduction of the heart rate may help prevent or alleviate the symptoms of the disease.

According to one aspect of the present invention there is provided a method of treating a disease, disorder or pathological condition in a patient in need of such treatment, the method comprising the step of administering to said patient a therapeutically effective amount of a polypeptide having the amino acid sequence of SEQ ID No.3, or an amino acid sequence having at least 60% sequence identity to SEQ ID No.3.

In another aspect of the present invention, a method of reducing the heart rate of an animal is provided, the method comprising the step of administering to the animal a therapeutically effective amount of a polypeptide having the amino acid sequence of SEQ ID No.3, or an amino acid sequence having at least 60% sequence identity to SEQ ID No.3.

In a further aspect of the present invention, a pharmaceutical composition is provided, the composition comprising a polypeptide having the amino acid sequence of SEQ ID No.3, or an amino acid sequence of at least 60% sequence identity to SEQ ID No.3, and a pharmaceutically acceptable diluent, adjuvant or carrier.

In yet a further aspect of the present invention packaging is provided, the packaging containing a pharmaceutical composition according to the present invention and instructions for use of said composition in a method of medical treatment.

In yet another aspect of the present invention an antagonist of the β₁- and/or β₂-adrenergic receptor is provided, the antagonist comprising a polypeptide having the amino acid sequence of SEQ ID No.3, or an amino acid sequence of at least 60% sequence identity to SEQ ID No.3.

In a further aspect of the present invention a pharmaceutical composition comprising a polypeptide having the amino acid sequence of SEQ ID No.3, or an amino acid sequence having at least 60% sequence identity to SEQ ID No.3, and a pharmaceutically acceptable diluent, adjuvant or carrier is provided for use in a method of medical treatment.

In yet another aspect of the present invention the use of a polypeptide having the amino acid sequence of SEQ ID No.3, or an amino acid sequence having at least 60% sequence identity to SEQ ID No.3 in the manufacture of a medicament for the treatment of a disease, disorder or pathological condition is provided.

Polypeptides according to the present invention may consist of, or comprise, any one of SEQ ID No.s 2, 3, 5, 6, 8, 9, 11, 12, 14, 15, 17, 18 or a polypeptide having at least 60% amino acid sequence identity, more preferably at least 65%, 70%, 75%, 80%, 85%, 86%, 87%, 88%, 89%, 90%, 91%, 92%, 93%, 94%, 95%, 96%, 97%, 98%, 99% or 100% sequence identity. Such polypeptides preferably have a minimum sequence length of at least 20 amino acids, more preferably at least 40 amino acids, and a maximum sequence length of no more than 90 amino acids. More preferably the polypeptides are between between 50 and 70 amino acids in length and are preferably one of 55, 56, 57, 58, 59, 60, 61, 62, 63, 64, 65, 66, 67, 68, 69, 70, 71, 72, 73, 74 or 75 amino acids in length.

FIG. 2 shows an alignment and comparison of cardiotoxins from O.hannah: SEQ ID No.s 3 (β-cardiotoxin), 6, 9, 12, 15 and 18. The Figure shows complete (100%) amino acid sequence identity of SEQ ID No.s 6, 9, 12, 15 and 18 with β-cardiotoxin (SEQ ID No.3) over 8 regions of each polypeptide (dark shading), including proposed loop regions. Small differences between each sequence are evident at the position of 7 single amino acids (no shading). These latter regions represent variable regions, in which substitution of one or more amino acids (of any kind) may be tolerated without loss of the functional properties of the polypeptide (e.g. reducing heart rate and/or β-adrenergic receptor antagonist activity).

As such the polypeptides in the present invention preferably consist of, or comprise, the sequence:

(SEQ ID No. 19)

RKCLNTPLPLIYX₁TCPIGQDX₂CX₃KMTIKKLPSKYDVIRGCX₄DICPK
SSADVX₅VX₆CCDTNKCX₇K,

which contains the sequences:

| | |
|---|---|
| RKCLNTPLPLIY | (SEQ ID No. 20) |
| X₁ | |
| TCPIGQD | (SEQ ID No. 21) |
| X₂CX₃ | (SEQ ID No. 22) |
| KMTIKKLPSKYDVIRGC | (SEQ ID No. 23) |
| X₄ | |
| DICPKSSADV | (SEQ ID No. 24) |
| X₅VX₆ | (SEQ ID No. 25) |
| CCDTNKCX₇K | (SEQ ID No. 26) |

or a sequence having at least 80% sequence identity to SEQ ID No.19, wherein each of X₁—X₇ is chosen from: any single amino acid; a contiguous sequence of 2, 3, 4, 5, 6, 7, 8, 9 or 10 amino acids of any type and in any combination, and wherein each of X₁—X₇ may be the same or different.

More preferably a polypeptide according to the present invention comprises an amino acid sequence having a plurality of contiguous sequence components each of those sequence components having at least 80% sequence identity with one of SEQ ID No.s 20, 21, 23, 24 and 26, and preferably being (i) of the same respective length; (ii) 1 or 2 amino acids longer; or (iii) 1 or 2 amino acids shorter. The sequence components will preferably be in corresponding linear position in the polypeptide amino acid sequence, as compared with the linear position of SEQ ID No.s 20, 21, 23, 24 and 26 in the polypeptide of SEQ ID No.19. More preferably, the polypeptide will have at least 3 such contiguous sequence components, still more preferably 4 or 5 such components. The percentage sequence identity may more preferably be chosen from 85%, 86%, 87%, 88%, 89%, 90%, 91%, 92%, 93%, 94%, 95%, 96%, 97%, 98%, 99% or 100%. The polypeptide may further preferably include one or more or each of SEQ ID No.s 22, 25, X₁ and X₄.

In any of these preferred embodiments, each of X₁—X₇ may be chosen from: any single amino acid, or a contiguous sequence of 2, 3, 4, 5, 6, 7, 8, 9 or 10 amino acids of any type and in any combination, and wherein each of X₁—X₇ may be the same or different.

Preferably in any such preferred embodiment, X₁ is a single amino acid chosen from T or K; X₂ is a single amino acid chosen from K or R; X₃ is a single amino acid chosen from V or I; X₄ is a single amino acid chosen from I or T; X₅ is a single amino acid chosen from E or V; X₆ is a single amino acid chosen from L or V; X₇ is a single amino acid chosen from N or D. The present invention includes any such combination of amino acids at positions X₁ to X₇.

Polypeptides of the invention may include or exclude one or more of SEQ ID No.s 46, 47 or 48.

The polypeptide as administered to a patient, e.g. in a pharmaceutical composition, may contain an N-terminal amino acid signal sequence, which may be cleaved (in vivo or ex vivo) to form a therapeutically active polypeptide. The signal sequence is preferably between 15 and 30 amino acids in length, more preferably between 18 and 25 amino acids, still more preferably one of 18, 19, 20, 21, 22, 23, 24 or 25 amino acids in length. The administered polypeptide may therefore be between 70 and 90 amino acids in length, more preferably one of 79, 80, 81, 82, 83, 84, 85, 86, 87, 88 or 89 amino acids in length.

It is generally preferred that the therapeutically active polypeptide, as formulated for administration, does not contain such a cleavable signal sequence. The administered polypeptide is then preferably between 50 and 70 amino acids in length, more preferably one of 55, 56, 57, 58, 59, 60, 61, 62, 63, 64, 65, 66, 67 or 68 amino acids in length.

The nucleic acids encoding polypeptides according to this invention also form a part of the present invention, including their use in producing polypeptides for use in therapeutic applications according to this invention. Nucleic acids having nucleotide sequences encoding each of the polypeptides of SEQ ID No.s 2, 3, 5, 6, 8, 9, 11, 12, 14, 15, 17 or 18 are shown in SEQ ID No.s 1, 4, 7, 10, 13 and 16, each forming part of the present invention. Corresponding sequences also encoding the same respective polypeptides, but differing in their exact sequence owing to the degeneracy of the genetic code, are similarly included in the present invention. Such nucleic acids may find particular use in the production of polypeptides for therapeutic uses according to this invention.

Accordingly, in a further aspect of the present invention a method of producing a polypeptide according to this invention is provided which is for use in treatment of a disease, disorder or pathological condition in a patient in need of such treatment, the method comprising introducing to a selected cell a nucleic acid having a nucleotide sequence encoding a polypeptide according to this invention such that said polypeptide is expressed in said cell; and harvesting the expressed polypeptide. Preferably, the cells are bacterial, yeast or mammalian cells. The nucleic acid may be preferably introduced on a suitable expression vector, e.g. plasmid expression vector. The harvested polypeptide may then be further processed and packaged, optionally including the step of mixing said polypeptide with a pharmaceutically acceptable carrier, diluent, adjuvant or other excipient to form a pharmaceutical composition.

The polypeptides according to this invention are preferably capable of reducing the heart rate in an intact animal, most preferably a mammal. The process of slowing the rate at which the heart beats is sometimes referred to as bradycardia.

The polypeptides of the invention are preferably capable of binding to a mammalian, preferably human, β₁- and/or β₂-adrenergic receptor. The binding is preferably specific, and is preferably antagonist binding resulting in antagonist action or antagonist activity of the polypeptide. Accordingly, polypeptides according to the present invention are useful in methods of treatment involving antagonist action of the polypeptide at β₁-adrenergic receptors and/or β₂-adrenergic receptors in the patient to be treated.

In this specification the term “antagonist” incorporates a molecule that is capable of blocking, disrupting or preventing the ability of a given chemical (e.g. ligand) to bind to its receptor, preventing a normal biological response; and/or a molecule that in binding to the receptor induces a biological response that is not normally associated with binding of that receptor with its cognate ligand. As such, in this specification a polypeptide may exhibit “antagonist action” and/or “antagonist activity” by blocking, disrupting or preventing the binding of a β-adrenergic receptor with its cognate ligand, and/or by binding of that polypeptide to a β-adrenergic receptor and provoking a biological response that is different to the response normally associated with binding of the receptor to its cognate ligand. In particular, polypeptides according to this invention may be capable of antagonist binding to β₁-adrenergic receptors thereby causing a reduction in heart rate.

Accordingly, in aspects according to this invention the polypeptide is preferably capable of exhibiting antagonist action at β₁- and/or β₂-adrenergic receptors.

Polypeptides according to this invention preferably have a low K₁ for the β₁- and/or β₂-adrenergic receptors—in the μM or nM range. Preferably, the polypeptides have a K₁ of less than 50 μM, more preferably less than 40 μM, 30 μM, 20 μM or 10 μM. The Kᵢ may be between 10 nM and 50 μM, more preferably between 10 nM and 30 μM, still more preferably between 100 nM and 20 μM or between 100 nM and 10 μM. The Kᵢ of SEQ ID No.3 for the β₁-adrenergic receptor is about 10 μM. The Kᵢ of SEQ ID No.3 for the β₂-adrenergic receptor is about 5 μM.

Polypeptides, and fragments thereof, according to this invention are preferably members of the three-finger toxin (3FTX) family.

The polypeptides according to the present invention have been demonstrated to be capable of reducing the mammalian heart rate and are useful in treating disorders by decreasing the heart rate of the patient. Accordingly, these polypeptides are useful in the treatment of a wide range of cardiovascular diseases (CVD). Such uses include treatment of tachycardia (being characterized by an abnormally rapid beating of the heart, e.g. a resting heart rate of over 100 beats per minute), including fetal tachycardia, cardiac arrhythmia, heart disease, heart failure, myocardial infarction, digitalis intoxication, use as an antihypertensive to treat hypertension (high blood pressure). Such uses may involve the antagonism of the cardiac β₁-adrenergic receptor.

The polypeptides are also considered useful in treatment of migraine, essential tremor, situational anxiety, alcohol withdrawal, hyperparathyroidism, glaucoma, portal hypertension and gastrointestinal bleeding.

β₃-cardiotoxin shows higher affinity to β₂-adrenergic receptors, which are the only subtype of β-ARs to be expressed on the membranes of major cell types in the skin like keratinocytes, fibroblasts and melanocytes. Given the findings by Pullar, β-cardiotoxin or peptides engineered from the same may be useful in the acceleration of wound healing.

Pharmaceutical compositions or medicaments according to the present invention may be formulated according to methodology well known to persons of skill in the art. For example, a polypeptide according to the present invention may be provided in liquid or gel formulation together with suitable pharmaceutical excipients and stabilising agents.

Polypeptides according to the present invention may be provided in pharmaceutically relevant quantities by expression of the polypeptide by well known recombinant gene technology methodology. For example, nucleic acid encoding the polypeptide, e.g. one of SEQ ID No.s 1, 4, 7, 10, 13 or 16 is cloned into a suitable plasmid expression vector and suitable bacterial, yeast or mammalian cells are transfected with said vector in order to express recombinant protein which may be harvested. Alternatively, polypeptides according to the present invention may be synthesised de novo by chemical polypeptide synthesis techniques.

### Polypeptide Components

As regards the subject-matter of the present invention the terms “peptide” and “polypeptide” are to be given the same meaning and are used interchangeably herein.

Whilst polypeptides used in the methods of the present invention may comprise full-length protein sequences, this is not always necessary. As an alternative, homologues, mutants, derivatives or fragments of the full-length polypeptide may be used, provided such alternative polypeptides retain the relevant functional characteristics, e.g. reducing heart rate and/or β-adrenergic receptor antagonist activity.

Derivatives include variants of a given full length protein sequence and include naturally occurring allelic variants and synthetic variants which have substantial amino acid sequence identity to the full length protein.

Protein fragments may be up to 10, 20, 30, 40, 50, 60 amino acid residues long. Minimum fragment length may be 8, 9, 10, 11, 12, 13, 14, 15, 16, 17, 18, 19, 20 or 30 amino acids or a number of amino acids between 3 and 30.

Mutants may comprise at least one modification (e.g. addition, substitution, inversion and/or deletion) compared to the corresponding wild-type polypeptide. The mutant may display an altered activity or property, e.g. binding.

Derivatives may also comprise natural variations or polymorphisms which may exist between individuals or between members of a family. All such derivatives are included within the scope of the invention. Purely as examples, conservative replacements which may be found in such polymorphisms may be between amino acids within the following groups:
(i) alanine, serine, threonine;
(ii) glutamic acid and aspartic acid;
(iii) arginine and leucine;
(iv) asparagine and glutamine;
(v) isoleucine, leucine and valine;
(vi) phenylalanine, tyrosine and tryptophan.

### Mimetics

Peptide mimetics of the polypeptides of the present invention form a further aspect of the present invention and may find use in the aspects and embodiments described herein. Such mimetics may be wholly synthetic compounds or may be the result of chemical and/or structural modification of the existing polypeptide structure. Methodology for designing suitable mimetics is described below. The resulting mimetics can be readily tested for their effect on the mammalian heart rate and/or binding and/or antagonism of β-adrenergic receptors.

The designing of functionally equivalent mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. some peptides may be unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

### Routes of Administration

Medicaments and pharmaceutical compositions according to aspects of the present invention may be formulated for administration by a number of routes, including but not limited to, topical, parenteral, systemic, intravenous, intra-arterial, intramuscular, subcutaneous, transdermal, oral and nasal. The medicaments and compositions may be formulated in fluid or solid form. Fluid formulations may be formulated for administration by injection to a selected region of the human or animal body. Injectable formulations may comprise the selected compound in a sterile or isotonic medium.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

A pharmaceutical composition according to this invention may be administered alone or in combination with other treatments, either simultaneously or sequentially dependent upon the condition to be treated.

### Patient

The patient to be treated may be any animal or human. The patient is preferably a mammal, more preferably a human patient. The patient may be male or female.

### Sequence Identity

Aspects of the present invention concern compounds which are isolated peptides/polypeptides comprising an amino acid sequence having a sequence identity of at least 60% with a given sequence. Alternatively, this identity may be any of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity.

Percentage (%) sequence identity is defined as the percentage of amino acid residues in a candidate sequence that are identical with residues in the given listed sequence (referred to by the SEQ ID No.) after aligning the sequences and introducing gaps if necessary, to achieve the maximum sequence identity, and not considering any conservative substitutions as part of the sequence identity. Sequence identity is preferably calculated over the entire length of the respective sequences.

Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence.

For example, where a given sequence comprises 100 amino acids and the candidate sequence comprises 10 amino acids, the candidate sequence can only have a maximum identity of 10% to the entire length of the given sequence. This is further illustrated in the following example:

| | | |
|---|---|---|
| (A) | | |
| Given seq: | XXXXXXXXXXXXXXX | (15 amino acids) |
| Comparison seq: | XXXXXYYYYYYY | (12 amino acids) |

% sequence identity=the number of identically matching amino acid residues after alignment divided by the total number of amino acid residues in the longer given sequence, i.e. (5 divided by 15)×100=33.3%

Where the comparison sequence is longer than the given sequence, sequence identity may be determined over the entire length of the given sequence. For example:

| | | |
|---|---|---|
| (B) | | |
| Given seq: | XXXXXXXXXX | (10 amino acids) |
| Comparison | XXXXXYYYYYYZZYZZZZZZ | (20 amino acids) |
| seq: | | |

% sequence identity=number of identical amino acids after alignment divided by total number of amino acid residues in the given sequence, i.e. (5 divided by 10)×100=50%.

Alignment for purposes of determining percent amino acid sequence identity can be achieved in various ways known to a person of skill in the art, for instance, using publicly available computer software such as BLAST, BLAST-2, ALIGN or Megalign (DNASTAR) software.

The invention includes the combination of the aspects and preferred features described except where such a combination is clearly impermissible or expressly avoided.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1. Abundance of various genes from cDNA library. Pie diagram shows the major groups of genes found in the library and their abundance. Inset, abundance of various novel toxin genes in the library.

FIG. 2. Multiple sequence alignment of novel proteins. A) A comparison of β-cardiotoxin with related sequences from O. hannah venom. B) A comparison of β-cardiotoxin with selected conventional CTXs reported from Naja sp. Changes at positions 23 and 53 are indicated by downward arrows. The homologous regions in CTXs from Naja sp. are shaded in grey. C) Weak toxin DE-1 homologs. The homologous regions in weak toxins from O. hannah are shaded in grey. D) Muscarinic toxin like protein 3 (MTLP-3) homolog. E) Long neurotoxin 2 (LNTX-2) homolog. Toxin names, species and accession numbers are shown. Conserved residues in all sequences are highlighted in black. Disulfide bridges and loop regions are also shown.

FIG. 3. Mutations in the loop regions of β-cardiotoxin. A) Changes in residues between β-cardiotoxin and CTX V₄II from Naja mossambica are highlighted in red over the crystal structure of the CTX V₄II (PDB #1CDT). B) Changes in residues between β-cardiotoxin and other related sequences from O. hannah venom shown in FIG. 2A. The three loops have been marked using Roman numbers. The image was generated using the DS Viewer Pro software.

FIG. 4. Purification of β-cardiotoxin. A) Gel filtration of O. hannah venom. Crude venom (100 mg/ml) was fractionated using a Superdex 30 Hiload (16/60) column. The column was pre-equilibrated with 50 mM Tris-HCl buffer (pH 7.4). Proteins were eluted at a flow rate of 1 ml/min using the same buffer. A downward arrow at peak 5 indicates the fractions containing β-cardiotoxin. B) RP-HPLC of peak 5 from gel filtration. Jupiter C₁₈ (5μ, 300 Å, 10 mm×250 mm) semi preparative column was equilibrated with 0.1% (v/v) TFA. The protein of interest was eluted from the column with a flow rate of 1.5 ml/min with a gradient of 31-42% buffer B (80% acetonitrile in 0.1% TFA). The downward arrow at peak 5d indicates the fractions containing β-cardiotoxin. C) ESI-MS of β-cardiotoxin. Reconstructed mass spectrum of β-cardiotoxin. CPS, counts/s; amu, atomic mass units.

FIG. 5. Comparison of secondary structure of β-cardiotoxin with conventional CTXs. A) Comparison of far-UV CD spectra. β-Cardiotoxin ( [symbol] ) naniproin ( [symbol] ) and cardiotoxin CM18 ( [symbol] ) were dissolved in MiiliQ water (0.5 mg/ml) and their CD spectra were recorded using a 0.1 cm path-length cuvette. B) Thermal denaturation of CM18. The protein was dissolved in MilliQ water (0.5 mg/ml) and far-UV CD spectra were recorded using a 0.1 cm path-length cuvette at 5° C. ( [symbol] ), 35° C. ( [symbol] ), (65° C. ( [symbol] ), 75° C. ( [symbol] ), 85° C. ( [symbol] ) and 95° C ( [symbol] ). Inset, CD spectra at 5° C. ( [symbol] ), 95° C. ( [symbol] ) and 5° C. (after cooling from 95° C.) ( [symbol] ). C) Thermal denaturation of β-cardiotoxin. The protein was dissolved in MilliQ water (0.5 mg/ml) and far-UV CD spectra were recorded using a 0.1 cm path-length cuvette. All the temperatures and notations are same as in B.

FIG. 6. Effect of temperature on tertiary structure of β-cardiotoxin. A) Near-UV CD spectroscopy. β-Cardiotoxin was dissolved in MilliQ water (1 mg/ml) and near-UV CD spectra were recorded using a 0.1 cm path-length cuvette. All the temperatures and notations are same as in FIG. 5B. B) Near-UV CD spectra at 5° C. ( [symbol] ), 95° C. ( [symbol] ) and 5° C. (after cooling from 95° C.) ( [symbol] ).

FIG. 7. Effect of β-cardiotoxin on blood coagulation and erythrocytes. A) Anticoagulant activity monitored by recalcification time (RT) and prothrombin time (PT) assays. Control (SO mM Tris-HCl buffer pH 7.4 added to the assay) clotting time is represented by the grey bars and for 50 μM β-cardiotoxin by black bars. Each data represents the mean±S.D. (n=3). B) Hemolytic activity of β-cardiotoxin and CM18. Control (0.9% NaCl) hemolytic activity is represented by the dotted bar and for various concentrations of β-cardiotoxin and CM18 by black and grey bars, respectively. Each data represents the mean±S.D. (n=3).

FIG. 8. Effects of β-cardiotoxin on cardiac function. Charts showing changes in ECG recorded before (above) and 10 min after (below) the administration of A) control (0.9% NaCl), B) 1 mg/kg CM18 and C) 1 mg/kg β-cardiotoxin. The horizontal arrows highlight the changes in ECG patterns. D) Change in heart rate (BPM, beats/min) 10 min after the administration of control (0.9% NaCl dotted bar), CM18 (1 mg/kg grey bar) and β-cardiotoxin (1 mg/kg black bar). Each data represents the mean±S.D. (n=3). E) Dose dependent reduction of heart rate 10 min after the administration of β-cardiotoxin. Each data point represents the mean±S.D. (n=3).

FIG. 9. Effects of β-cardiotoxin on Langendorff perfused hearts. Charts showing changes in heart rate (HR) recorded before (above) and 10 min after (below) the treatment with A) control (KH solution) and B) 5 μM β-cardiotoxin dissolved in KH solution. C) Changes in HR 10 min after treatment with control (KH solution) shown by grey bar and 5 μM β-cardiotoxin shown by black bar. D) Changes in LVDEP (left ventricle diastolic end pressure) (mmHg) 10 min after treatment with control (KH solution) shown by grey bar and 5 μM β-cardiotoxin shown by black bar. The ±S.D. are indicated at the top of the box (n=3).

FIG. 10. Interaction of β-cardiotoxin with β-ARs.

Displacement of radiolabelled ligand (−)-[³H]CGP-12177 by (−)-Propranolol (Open symbols) and β-cardiotoxin (solid symbols) A) Interaction with cloned human β₁-AR and B) β₂-AR. Each data point represents the mean±S.D of two replicates.

FIG. 11. Prediction of secondary structure of β-cardiotoxin.

The secondary structure was predicted by submitting the protein sequence to the PORTER prediction tool available at http://distill.ucd.ie/porter/. The first line shows the β-cardiotoxin sequence and the bottom line indicates the predicted secondary structural elements. E—strand regions.

FIG. 12. O.hannah cardiotoxin nucleotide and amino acid sequences.

SEQ ID NO.s 1-3:

Nucleotide sequence encoding full length β-cardiotoxin from O. hannah (SEQ ID No.1; accession no. AY354198.1 GI:38049473); amino acid sequence of full length β-cardiotoxin encoded by SEQ ID No.1 (SEQ ID No.2; accession no. AAR10440.1 GI:38049474); and amino acid sequence of β-cardiotoxin following cleavage of the signal peptide from SEQ ID No.2 (SEQ ID No.3).

SEQ ID NO.s 4-6:

Nucleotide sequence encoding full length cardiotoxin from O. hannah (SEQ ID No.4; accession no. DQ273579.1 GI:82570096); amino acid sequence of full length cardiotoxin encoded by SEQ ID No.4 (SEQ ID No.5; accession no. ABB83633.1 GI:82570097); and amino acid sequence of cardiotoxin following cleavage of the signal peptide from SEQ ID No.5 (SEQ ID No.6).

SEQ ID NO.s 7-9:

Nucleotide sequence encoding full length cardiotoxin from O. hannah (SEQ ID No.7; accession no. DQ273577.1 GI:82570092); amino acid sequence of full length cardiotoxin encoded by SEQ ID No.7 (SEQ ID No.8; accession no. ABB83631.1 GI:82570093); and amino acid sequence of cardiotoxin following cleavage of the signal peptide from SEQ ID No.8 (SEQ ID No.9).

SEQ ID NO.s 10-12:

Nucleotide sequence encoding full length cardiotoxin from O. hannah (SEQ ID No.10; accession no. DQ273581.1 GI:82570100); amino acid sequence of full length cardiotoxin encoded by SEQ ID No.10 (SEQ ID No.11; accession no. ABB83635.1 GI:82570101); and amino acid sequence of cardiotoxin following cleavage of the signal peptide from SEQ ID No.11 (SEQ ID No.12).

SEQ ID NO.s 13-15:

Nucleotide sequence encoding full length cardiotoxin from O. hannah (SEQ ID No.13; accession no. DQ273578.1 GI:82570094); amino acid sequence of full length cardiotoxin encoded by SEQ ID No.13 (SEQ ID No.14; accession no. ABB83632.1 GI:82570095); and amino acid sequence of cardiotoxin following cleavage of the signal peptide from SEQ ID No.14 (SEQ ID No.15).

SEQ ID NO.s 16-18:

Nucleotide sequence encoding full length cardiotoxin from O. hannah (SEQ ID No.16; accession no. DQ273580.1 GI:82570098); amino acid sequence of full length cardiotoxin encoded by SEQ ID No.16 (SEQ ID No.17; accession no. ABB83634.1 GI:82570099); and amino acid sequence of cardiotoxin following cleavage of the signal peptide from SEQ ID No.17 (SEQ ID No.18).

Accession numbers are for the NCBI database:

http://www.ncbi.nlm.nih.gov/

FIG. 13. Crystallization of β-cardiotoxin.

A) β-Cardiotoxin crystals obtained in 0.1M SPG buffer pH 7, containing 25% PEG 1500 and 0.1 μl of 0.1 M spermidine. Protein concentration used was 15 mg/ml. B) X-ray diffraction pattern of β-cardiotoxin crystals obtained at the in house X-ray generator.

FIG. 14. Design of short peptides.

Short peptides based on β-cardiotoxin sequence were designed from the native β-cardiotoxin sequence (SEQ ID No.3). BCL1a (SEQ ID No. 46) and BCL1b (SEQ ID No. 47) are based on the ‘Proline-bracket hypothesis’ proposed by Kin and Evans. The single Cys in the native sequence has been changed to Ala in BCL1a and BCL1b (highlighted in black). The hypothetical functional sites have been highlighted using boxes. BCL2 (SEQ ID No.48) was cyclised by oxidation of the two Cys residues.

FIG. 15. Purification and characterization of the short peptides.

A), C) and E) RP-HPLC of synthesised peptides BCL1a, BCL1b and BCL2, respectively. Jupiter C₁₈ (5μ, 300 Å, 10×250 mm) semipreparative column was equilibrated with 0.1% (v/v) TFA. Peptide of interest was eluted from the column with a flow rate of 1.5 ml/min with a gradient of buffer B (80% acetonitrile in 0.1% TFA). Downward arrow at peaks indicates fractions containing the desired peptides. B), D) and F) ESI-MS of purified peptides BCL1a, BCL1b and BCL2, respectively. CPS counts/s.

FIG. 16. Interaction of peptides with β-ARs.

Displacement of radiolabeled ligand (−)-[³H]CGP-12177 by (−)-propranolol (Blue line), BCL1a (), BCL1b (∘) and BCL2 (♦). A), B) and C) show interaction between β1AR and BCL1a, BCL1b and BCL2, respectively. D), E) and F) show interaction between β2AR and BCL1a, BCL1b and BCL2, respectively. Each data point represents mean of 2 replicates.

### DETAILED DESCRIPTION OF THE INVENTION

Specific details of the best mode contemplated by the inventors for carrying out the invention are set forth below, by way of example. It will be apparent to one skilled in the art that the present invention may be practiced without limitation to these specific details.

### Example 1

In an effort to exploit the rich miscellany of snake venoms, we constructed a partial cDNA library using mRNA extracted from Ophiophagus hannah venom gland tissue and identified five genes coding for proteins belonging to the 3FTX family. We report the identification, purification and characterization of one of these novel proteins, β-cardiotoxin from the venom of O. hannah. This protein differs from classical cardiotoxins in their structure as well as function. This is the first report of an exogenous protein targeting the β-adrenergic receptor (AR) system causing a marked reduction in heart rates in whole animals as well as isolated perfused rat hearts. Thus, we named this novel member of the 3FTX family as β-cardiotoxin. We also describe the identification of a unique folding intermediate of β-cardiotoxin and its implications in protein folding.

### Experimental Procedures

### Materials

Ophiophagus hannah venom glands were frozen in liquid nitrogen immediately after dissection and kept in −80° C. until use. The reagents and kits used in the cDNA library construction and screenings were as follows: restriction endonucleases (New England Biolabs®, Beverly, Mass.), pGEMTeasy vector (Promega, Madison, Wis.), RNeasy® Mini kit and QlAprep® Miniprep kit (Qiagen GmbH, Hilden, Germany), SMART™ RACE cDNA amplification kit (Clontech Laboratories Inc., Palo Alto, Calif.), ABI PRISM® BigDye™ Terminator Cycle Sequencing Ready Reaction Kit (Version 3.0) (PE-Applied Biosystems, Foster City, Calif.) and Luria Bertani broth and agar (Q.BIOgene, Irvine, Calif.).

Lyophilized O. hannah venom was obtained from Mr. Duncan MacRae (Bali, Indonesia). All chemicals were purchased from Sigma-Aldrich except the following: reagents for Edman degradation N-terminal sequencing (Applied Biosystems, Foster City, Calif.), and acetonitrile (Merck KGaA, Darmstadt, Germany). Superdex 30 Hiload (16/60) column and Jupiter C18 (5μ, 300 Å, 10 mm×250 mm) were purchased from Amersham Biosciences (Uppsala, Sweden) and Phenomenex (Torrance, Calif.), respectively. Water was purified using a MilliQ system (Millipore, Billerica, Mass.). All chemical reagents used were of the highest purity available.

### Animals

Swiss albino male mice (˜20 g) were used for the in vivo toxicity study. Male Sprague-Dawley (SD) rats were used for electrocardiogram (ECG) monitoring (3-4 weeks old, ˜80-100 g) and the Langendorff isolated perfused heart experiments (8 weeks old, ˜250 g). Animals were acquired from the university Laboratory Animal Center (LAC) and were acclimatized to the Animal Holding Unit surroundings for at least 3 days prior to the experiments. Animals were kept under standard conditions with food and water available ad lib. All animal experiments were conducted according to the protocol (776/05a) approved by the Institutional Animal Care and Use Committee (IACUC) of the National University of Singapore.

### Isolation of Total RNA

Total RNA was isolated from O. hannah venom gland using RNeasy® Mini kit. For each extraction, 30 mg venom gland tissue was first pulverized in liquid nitrogen using a cooled mortar and pestle and further homogenized for 20 to 30 s using a Heidolph DIAX₆₀₀ homogenizer (Schwabach, Germany) in the presence of 600 μl Buffer RLT. The integrity of the RNA extracted was examined by denaturing agarose gel electrophoresis.

cDNA Library Construction

The 5′-RACE ready cDNA was obtained using a SMART™ RACE cDNA amplification kit from Clontech. The BD SMART II™A oligonucleotide and an oligo (dT) primer supplied with the kit were used for this purpose. The double stranded cDNA for cloning was obtained using the long-UP and oligo (dT) primers provided in the kit. The double stranded cDNA was cloned into pGEMT-easy vector system using the TA cloning approach and the plasmids were introduced into competent E. coli DH5α cells by heat shock transformation.

Sequencing of cDNA Clones

Transformed clones were selected on a plate containing IPTG and X-gal by bluewhite selection. Plasmids were extracted from overnight cultures using QIAprep® Miniprep kit, digested using EcoRI and fractionated on a 1% agarose gel to confirm the presence of the insert. Plasmids having the inserts were sequenced separately using T7 and SP6 primers and the full-length sequences were assembled and analyzed.

### Purification of the Protein

O. hannah crude venom (100 mg in 1 ml of MilliQ water) was loaded onto a Superdex 30 (16/60) gel filtration column that was equilibrated with 50 mM Tris-HCl buffer (pH 7.4) and eluted with the same buffer using an AKTA purifier system (Amersham Biosciences, Uppsala, Sweden). The eluted samples were pooled into eight fractions. These were further sub-fractionated by reverse phase-high performance liquid chromatography (RP-HPLC) using a Jupiter C18 (5μ, 300 Å, 10 mm×250 mm) column that was equilibrated with 0.1% trifluoroacetic acid (TFA) and the proteins eluted with a linear gradient of 80% acetonitrile in 0.1% TFA. The fractions were collected and directly injected into an API-300 liquid chromatography/tandem mass spectrometry system (PerkinElmer Life Sciences) for mass determination. Fractions showing the expected molecular mass were pooled and lyophilized.

### Electrospray Ionization Mass Spectrometry

The mass and homogeneity of the novel protein was determined by electrospray ionization mass spectrometry (ESI-MS) using an API-300 liquid chromatography/tandem mass spectrometry system (PerkinElmer Life Sciences). RP-HPLC fractions were directly used for the analysis. Ion spray, orifice and ring voltages were set at 4600, 50 and 350 V, respectively. Nitrogen was used as the nebulizer and curtain gas. A Shimadzu LC-LOAD pump was used for solvent delivery (40% acetonitrile in 0.1% formic acid) at a flow rate of 40 μl/min. BioMultiview software (PerkinElmer Life Sciences) was used to analyze and deconvolute the raw mass data.

### N-Terminal Sequencing

N-terminal sequencing of the native protein was performed by automated Edman degradation using a PerkinElmer Applied Biosystems 494 pulsed-liquid phase protein sequencer (Procise) with an on-line 785A phenlythiohydantion (PTH)-derivative analyzer. The PTH-derivatized amino acids were then sequentially identified by mapping the respective separation profiles with the standard chromatogram.

### Circular Dichroism (CD) Spectroscopy

Far-UV CD spectra (260-190 nm) were recorded using a Jasco J-810 spectropolarimeter (Jasco Corporation, Tokyo, Japan). All measurements were carried out at room temperature using a 0.1 cm path length capped cuvette. The instrument optics and cuvette chamber were continuously flushed with 30 l of nitrogen/min before and during the recording of the spectra. The spectra were recorded using a scanning speed of 50 nm/min, a resolution of 0.1 nm and a bandwidth of 1 nm. A total of three scans were recorded and averaged for each spectrum, and the baseline was subtracted. All samples were dissolved in MilliQ water.

### Thermal Denaturation Studies Using CD Spectroscopy

A Jasco J-810 spectropolarimeter connected to a Grant LTD6G water bath controlled by Jasco PTC-423S was used for this study. All recording parameters were the same as mentioned above. The temperature range was from 5° C. to 95° C. with a pitch of 10° C. Far-UV CD spectrum (260 to 190 nm) and near-UV spectrum (350 to 250 nm) were recorded after each 10° C. increase. The CD was also continuously monitored at 203, 222 and 280 nm with a data pitch of 0.1° C. and a bandwidth of 1 nm. The temperature slope used for the study was 5° C./min.

### Methods for Protein Administration

The protein was dissolved in 200 μl of 0.9% NaCl (Braun, Malaysia) and administered through the intra-peritoneal (i.p.) route for the in vivo toxicity study in mice. Intra-tail vein (i.t.v.) administration was done in the ECG monitoring study in rats. The various protein doses were dissolved in 200 μl of 0.9% NaCl solution and injected into the tail vein using a 27 G ½ needle (Becton Dickinson, Franklin Lakes, N.J.) held at an angle of about 10° to the tail. The infusion was made very slowly and at a steady rate.

### In Vivo Toxicity Study

After purification of the protein from snake venom, i.p. doses of 1 mg/kg, 10 mg/kg and 100 mg/kg were administered to healthy male mice (three animals per group) and the symptoms were observed and recorded. 0.9% NaCl was injected as control. Postmortem examinations were conducted on the animals after their death/sacrifice.

### Anticoagulant Activity

The anticoagulant activity of the purified protein was tested by two coagulation tests using a BBL fibrometer. Blood plasma was collected from healthy human volunteers.

Prothrombin time—The prothrombin times were measured according to the method of Quick (25). 100 μl of 50 mM Tris-HCl buffer (pH 7.4), 100 μl of plasma and 50 μl of the protein dissolved in the assay buffer were preincubated for 2 min at 37° C. Clotting was initiated by the addition of 150 μl of thromboplastin with calcium reagent (Sigma-Aldrich, St Louis, Mo.).

Recalcification time—The recalcification times were measured according to the method of Langdell et al. (26). 100 μl of 50 mM Tris-HCl buffer (pH 7.4), 100 μl of plasma and 50 μl of the protein dissolved in the assay buffer were preincubated for 2 min at 37° C. Clotting was initiated by the addition of 50 μl of 50 mM CaCl₂.

### Hemolytic Assay

Blood from healthy human volunteers was centrifuged for 15 min at 3000 rpm at room temperature. The plasma was discarded and RBC's were washed three times with 0.9% NaCl. The proteins were dissolved in 0.95 ml of 0.9% NaCl and 5 μl of washed RBC was added to obtain a 0.5% suspension. 0.9% NaCl was taken as the negative control. The tubes were incubated at 37° C. for 1 h. RBC were removed by centrifugation at 3000 rpm for 15 min at room temperature and the absorbance of the supernatant was read at 418 nm and 540 nm.

### Electrocardiogram Monitoring and Heart Rate Determination

For electrocardiogram (ECG) measurements, Sprague-Dawley rats were anesthetized with an i.p. dose of 7% chloral hydrate (5 ml/kg). Needle probes (29-gauge, MLA1204) were inserted into the front and back left paw pads of each mouse, and signals were captured using the Animal BioAmp differential amplifier (ML136). Signals were digitized using an eight-channel Powerlab 8SP (ML785), and recordings were displayed with Chart 5 software (ADInstruments, Castle Hill, NSW, Australia). All probes and equipment for ECG measurements were obtained from ADInstruments (Castle Hill, NSW, Australia). Continuous recording was made from 5 min before an i.t.v. injection of various doses of the protein until 20 min after the injection. The same volume of 0.9% NaCl was administered to the control group. The heart rate at different time points before and after the injection of the protein was estimated using the Chart 5 software.

### Isolated Perfused Heart

Rats were anesthetized with 7% chloral hydrate (5 ml/kg). 2 ml of 50 I.U. heparin (David Bull Laboratories, Warwick, UK) was injected i.p. 15 min prior to sacrificing the animal. The heart was excised rapidly and placed in oxygenated Krebs-Henseleit (KH) solution (in mM/1: NaCl 118.0, KCl 4.5, KH₂PO₄ 1.4, MgSO₄ 1.2, NaHCO₃ 25, CaCl₂ 1.4 and glucose 11; pH 7.4) before the aorta was cannulated on the Langendorff apparatus. Non-recirculating mode of retrograde perfusion with KH solution was carried out at a constant flow (6-7 ml/min) at 37° C. The buffer was continuously bubbled with 95% O₂ and 5% CO2. The hearts were allowed to equilibrate for 30 min and in study groups the buffer flow was switched to a second reservoir which contained 5 μM of the protein dissolved in KH solution, plain KH solution was passed for further 15 min in case of control group.

### Measurement of Isovolumetric Cardiac Performance

A water-filled latex balloon was attached to a pressure transducer and inserted through the mitral valve into the left ventricle (LV) through an incision in the left atrium. The pressure transducer was connected to a Powerlab (ADInstruments, Castle Hill, NSW, Australia) data recording system. The following indices of cardiac performance were measured and averaged from ten beats for each condition: heart rate (HR, in beats/min) and left ventricle diastolic end pressure (LVDEP, in mmHg), which is an index of contractile activity. Premature contractions were excluded from the analyses.

### Competitive Binding Assay

Cloned human β-adrenergic receptor subtypes 1 and 2 (βᵢ and β₂) produced in Sf9 cells were purchased from PerkinElmer Life and Analytical Sciences (Boston, Mass.). The radioligand (−)-[³H]CGP-12177 was purchased from GE Healthcare (Buckinghamshire, UK). For the competitive binding studies we used 0.15 nM (−)-[³H]CGP-12177, β₁ or β₂ and β-cardiotoxin ranging from 5 nM to 10 μM in 75 mM Tris-HCl buffer containing 12.5 mM MgCl₂ and 2 mM EDTA, pH 7.4. Total reaction volume was 1050 μl. Nonspecific binding was determined to be 2-3% for β₁ and 1-2% for β₂ by the inclusion of 2 μM (S)-(−)-propranolol hydrochloride. After 60 min incubation at room temperature, the reaction mixtures were filtered through Whatman GF/C glass microfibre filters (Maidstone, England) presoaked in ice cold wash buffer (50 mM Tris-HCl buffer, pH 7.4). The filters were washed nine times with 500 μl (each time) of ice cold wash buffer. A Beckman LS3801 liquid scintillation counter was used to measure the radioactivity retained on the washed filters.

The binding affinity Kᵢ was calculated from the IC₅₀ using the equation of Cheng and Prusoff (27),

Kᵢ=IC₅₀÷{1+([Radioligand]/K_{d})}

Where, the IC₅₀ (concentration of the inhibitor that displaces 50% of bound ligand) values were determined by plotting the % specific binding in the Y-axis versus log [molar concentration of protein used] in the X-axis, K_{d} is the binding affinity of the radioligand to the receptor.

### Results

cDNA Library Construction and Identification of Novel Toxin Genes

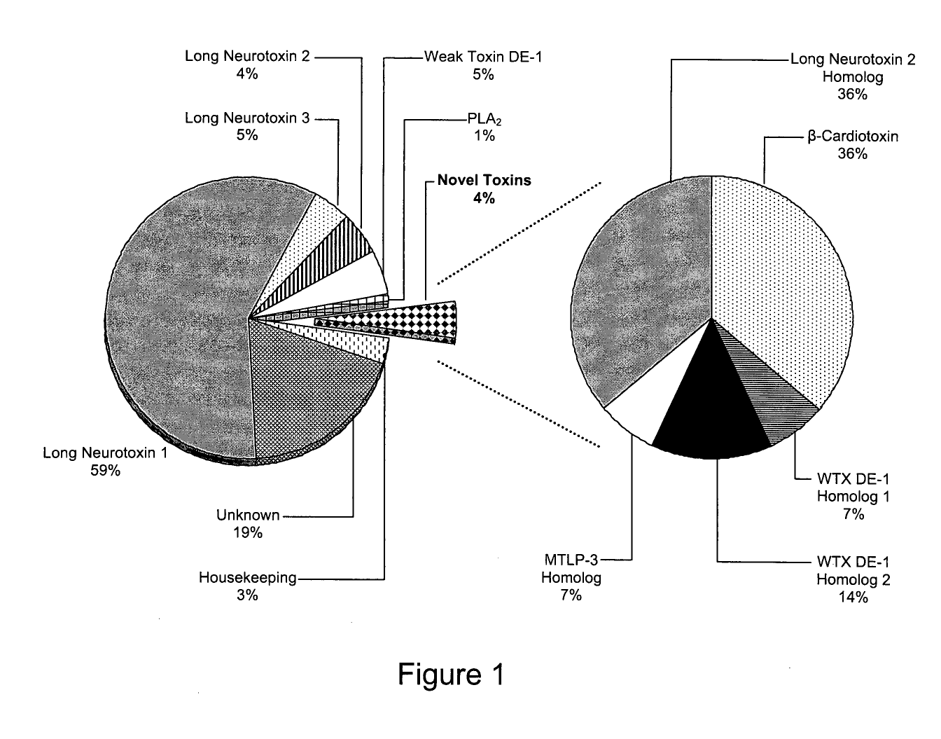

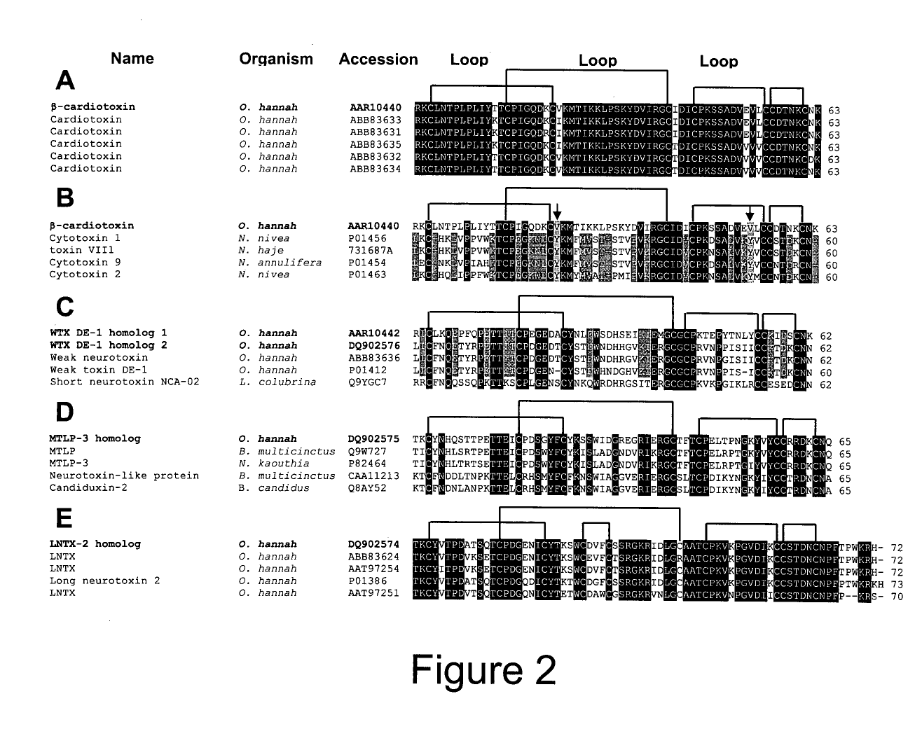

A partial cDNA library was constructed using mRNA isolated from venom gland tissue of the snake. As we were interested in low molecular weight proteins from the venom, we picked 346 clones from this library containing inserts ranging in size from 100 to 800 bp. The most abundant sequence found in the library was that of long neurotoxin 1 (FIG. 1). Five new toxin genes were identified and their fulllength sequences were submitted to the NCBI GenBank data base (AY354198, AY354200, DQ902574, DQ902575 and DQ902576). From the conserved Cys pattern we concluded that all these genes encoded for proteins belonging to the 3FTX family of snake venom proteins (FIG. 2). Two non-toxin genes belonging to the ribosomal complex were also isolated (AY354199 and AY357074). Several other sequences (19%) that were obtained did not show any match with available sequences from the data base (FIG. 1).

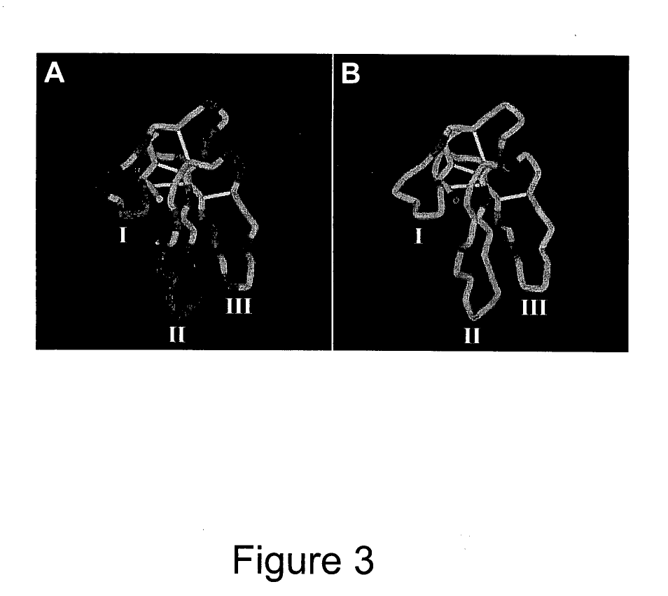

The cDNA AY354198 encodes for a protein with a molecular weight of 7012.42 Da, which is closely related to proteins reported as precursors of cardiotoxins (CTXs) (ABB83631, ABB83632, ABB83633, ABB83634 and ABB83635) all identified from O. hannah venom glands (FIG. 2A). These sequences are highly similar to each other, differing only at 2 to 5 sites out of which the substitutions K20R, V22I and L53V are conserved changes (FIG. 2A). Despite being classified as CTXs (or in one case as a short chain alpha neurotoxin (AAT97262)) the biological properties of none of these proteins have been characterized. Interestingly, they showed only 55-65% sequence identity with conventional CTXs and CTX-like proteins (FIG. 2B) and even poorer sequence identity with neurotoxins (data not shown). Further, most of the structural differences between AY354198 and conventional CTXs were found in the loop regions (FIG. 3A), which play an important role in the interaction of 3FTXs with various target proteins (13). Therefore, we expected this protein to interact with a distinct protein target and to exhibit different pharmacological effects than classical CTXs and hence examined the biological properties of this protein. As shown below, this protein indeed shows distinct biological properties compared to other snake venom CTXs and hence belongs to a new class of 3FTXs. Unlike conventional CTXs, it acts as a beta-blocker and decreases the heart rate and hence was named as β-cardiotoxin.

Isolation and Purification of β-cardiotoxin

Our initial LC/MS studies of O. hannah venom (28) showed that it contains a protein with the molecular weight of 7013.80±1.27 Da indicating the presence of AY354198 protein in the venom. The novel protein was purified by following the calculated mass of the protein while fractionating the crude venom of O. hannah using chromatographic techniques. We used a two-step chromatography approach, where the first step comprised of separating the venom components based on their sizes into eight peaks using gel filtration chromatography

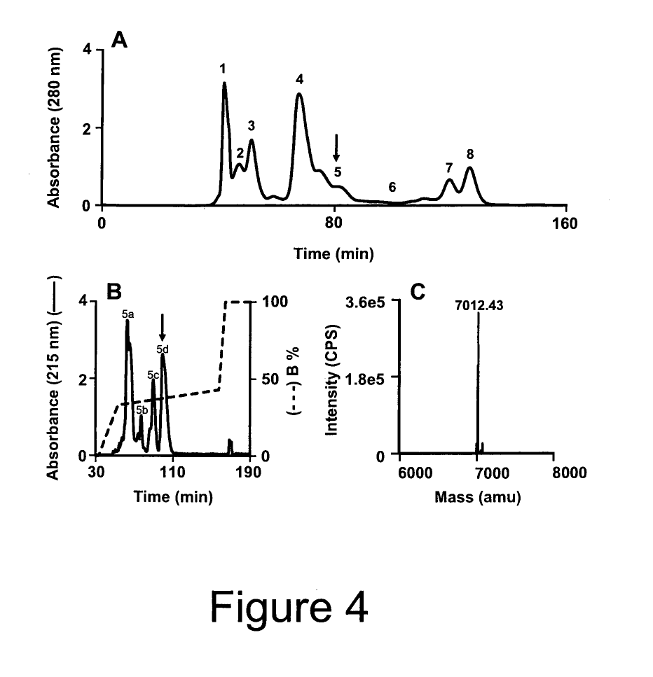

(FIG. 4A). Subsequently, each peak was fractionated by RP-HPLC using a Jupiter C18 semi preparative column (for example, peak 5 (FIG. 4B)). Each RP-HPLC fraction was then subjected to ESI-MS analysis to determine the mass and hence the tentative identification of the protein (data not shown). The ESI-MS of fraction 5d (FIG. 4B) showed three peaks with mass/charge ratios ranging from +4 to +6 charges (data not shown) and the final reconstructed spectrum showed a molecular weight of 7012.43±0.91 Da (FIG. 4C). The ESI-MS spectrum also revealed the purity of the protein (FIG. 4C). N-terminal Edman degradation sequencing of the first 30 residues further confirmed the identity of the protein.

### CD Spectroscopy

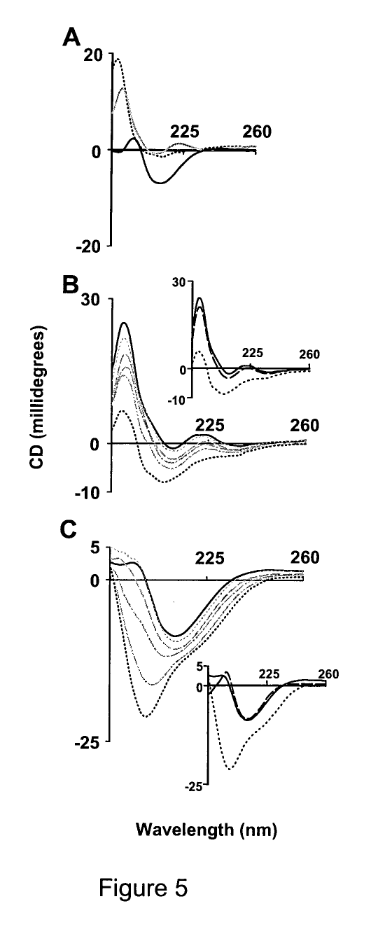

The secondary structural elements present in conventional cardiotoxins and β-cardiotoxin were analyzed using CD spectroscopy. Naniproin, a CTX isolated from the venom of Naja nigricollis², showed a small minimum at 215-220 nm and an intense maximum at 190-195 nm and CM18, a CTX from the venom of Naja atra, showed a maximum at 220-225 nm, a small minimum at 210-212 nm and an intense maximum at 190-195 nm (FIG. 5A). Interestingly, β-cardiotoxin showed intense minimum at 212-215 nm and small maximum at 198 nm and differed significantly from the CD spectra of the other two conventional CTXs (FIG. 5A).

### Thermal Denaturation Studies

CD spectroscopy was employed to study the thermal unfolding of CM18 and β-cardiotoxin. There was a marked decrease in the β-sheet signal for CM18 at 95° C. and the band at 212 nm shifted towards 190 nm indicating the induction of random coil conformation (FIG. 5B). Interestingly, β-cardiotoxin underwent a complete structural transition at higher temperatures. Double minima at 222-225 nm and 203 nm started to appear at 85° C. indicating the presence of α-helical secondary structural elements (FIG. 5C). Further, β-cardiotoxin was less thermostable compared to conventional CTX and started to undergo structural transition at about 75° C. (FIG. 5C). Both the proteins attained their native structures upon cooling to 5° C. (FIGS. 5, B and C, insets) showing that the thermal unfolding of both the proteins was completely reversible.

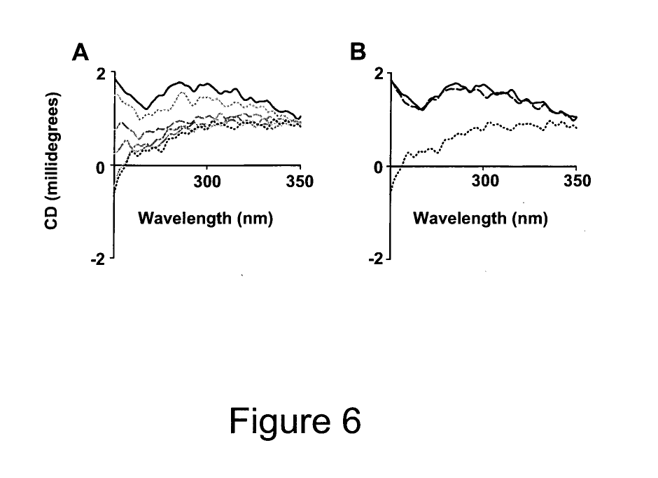

We further investigated the effect of temperature on the tertiary structure of β-cardiotoxin using near-UV CD spectroscopy. We used a higher concentration of protein (1 mg/ml) compared to far-UV CD experiments as the signals in this region are usually weak (29). Thermal denaturation experiments as described above were repeated with a wavelength scan from 350 nm to 250 nm. The weak positive signals obtained are probably due to the complete absence of Trp and Phe and the presence of only two Tyr residues in the protein. Interestingly, at higher temperatures (>75° C.) there was a drop in the signal implying a total loss of tertiary structure (FIG. 6A). When the sample was slowly cooled down to 5° C., there was a complete recovery in the CD signal showing that the process is reversible (FIG. 6B). The loss of tertiary structure observed in the near-UV spectrum (FIG. 6) coincided with the β-sheet to α-helix transition that was observed in the far-UV spectrum (FIG. 5C) revealing the presence of a ‘molten globule’-like state that retained secondary structure with concomitant loss of tertiary structure (30-32).

### In Vivo Toxicity Study

Three groups of three male mice (˜20 g) each were used in the toxicity studies. The first group received three increasing doses of β-cardiotoxin (1 mg/kg, 10 mg/kg and 100 mg/kg) and the ensuing effects were closely monitored. The protein was not lethal up to 10 mg/kg dosage. At 100 mg/kg, the mice showed symptoms of labored breathing, impaired locomotion, lack of response to external stimuli and death occurred after about 30 min. Postmortem examinations did not reveal any hemorrhage or visual damage to internal organs. To the second group, a single dose (2.5 mg/kg) of α-bungarotoxin, a potent neurotoxin isolated from the venom of Bungarus candidus was injected. These animals exhibited severe paralysis of the hind limbs and they died in about 10 min after the administration. To the last group, 0.9% NaCl was administered as a negative control and no symptoms developed until the animals were ultimately sacrificed at the end of the study.

### Anticoagulant Effects

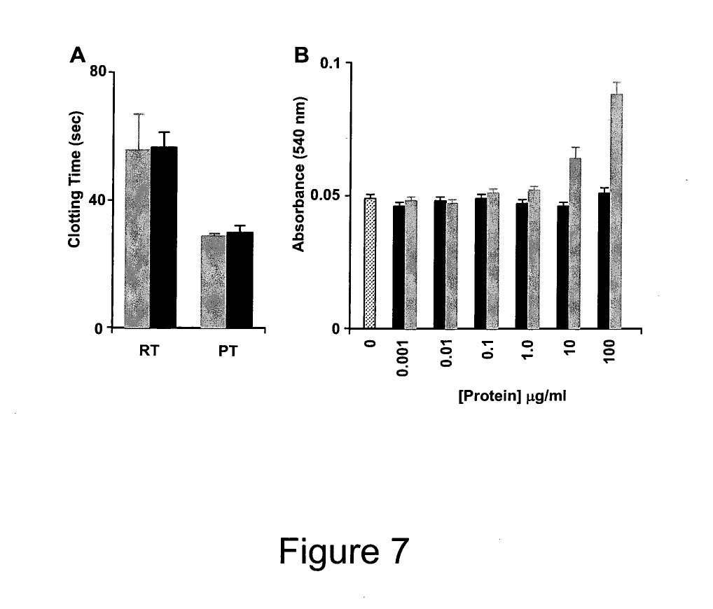

The effects of β-cardiotoxin (final concentration of 50 μM protein per assay) on the extrinsic and intrinsic pathways of blood coagulation were assessed using the prothrombin time and recalcification time, respectively. β-Cardiotoxin did not cause any significant changes in the clotting time compared to the control in both assays (FIG. 7A). Thus, we conclude that β-cardiotoxin does not affect blood coagulation.

### Hemolytic Activity

Washed human erythrocytes were treated with β-cardiotoxin at concentrations ranging from 1 ng/ml to 100 μg/ml (final concentration per assay) to determine the hemolytic activity. The protein did not show any significant hemolytic activity as the absorbance values of the highest doses were comparable to that of the negative control (FIG. 7B). In contrast, CTX CM18 showed significant hemolytic activity (FIG. 7B).

Cardiac Effects of β-cardiotoxin

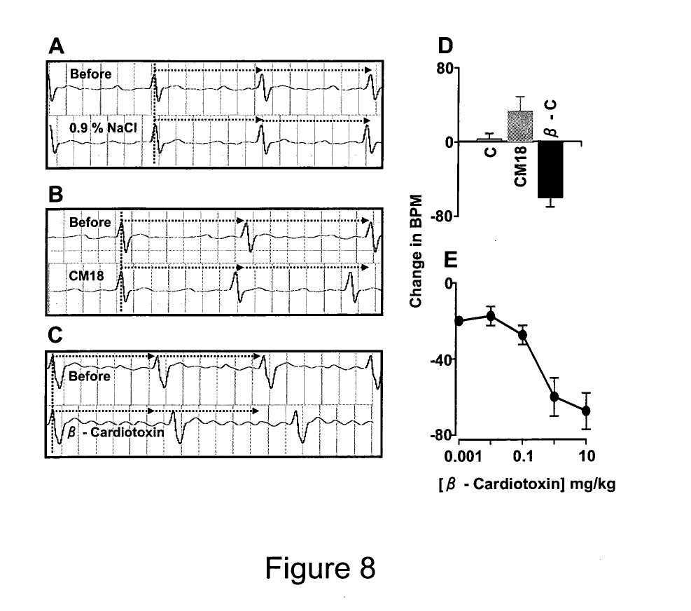

The in vivo activity of the protein was determined by administering the protein into anesthetized male rats and monitoring the changes induced in the ECG patterns. For the control animals the carrier alone (0.9% NaCl) was injected and there was no change in the heart rate after injection (FIG. 8A). The administration of CTX CM18, as expected, increased the heart rate and induced a positive chronotropic effect (FIG. 8B). In contrast, there was decrease in the heart rate as indicated by an increase in the distance between successive QRS complexes upon administration of β-cardiotoxin, suggesting a negative chronotropic effect that might cause bradycardia (FIG. 8C). Thus, a conventional CTX increases the heart rate, while β-cardiotoxin decreases the heart rate (FIG. 8D). This decrease in the heart rate induced by β-cardiotoxin is dose-dependent (FIG. 8E). Thus, β-cardiotoxin induces negative chronotropism in the heart rate in rats unlike conventional CTXs.

### Isolated Perfused Heart Studies

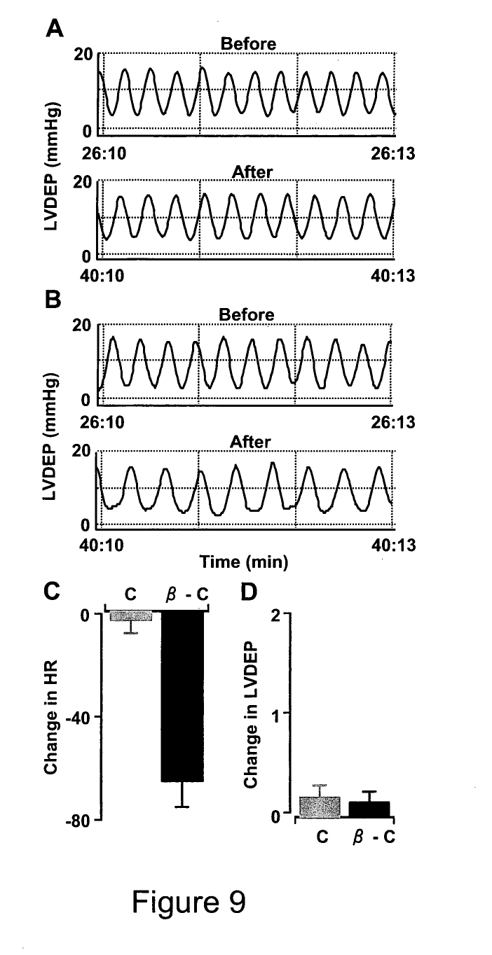

The direct effects on cardiac tissue were determined using the Langendorff isolated perfused rat hearts. There were no changes in any of the cardiac parameters in the control group (FIG. 9A), whereas β-cardiotoxin induced a negative chronotropic effect (FIG. 9B). β-Cardiotoxin at 5 μM caused a marked reduction in the heart rate (FIG. 9C) without any significant change in the contractility as indicated by the LVDEP (FIG. 9D). Thus, the decrease in the heart rate induced by β-cardiotoxin in rats is most probably due to its direct action on the cardiac muscles.

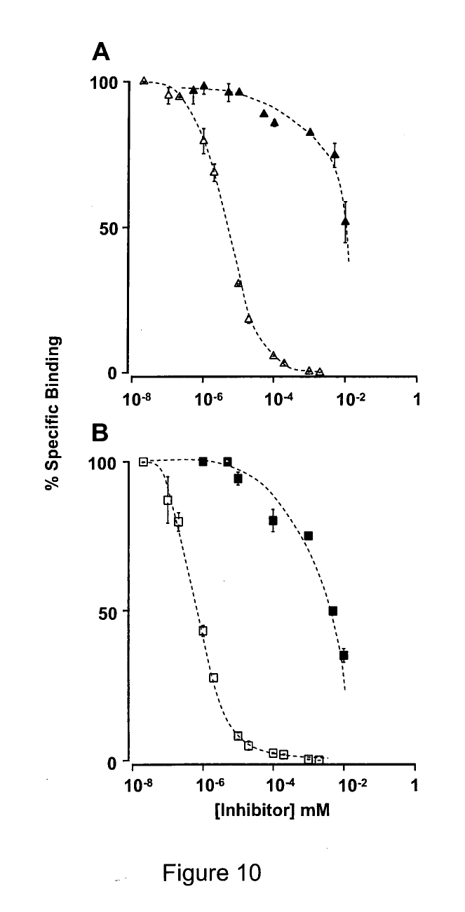

β-Cardiotoxin interaction with β-adrenergic receptors (β-ARs) β-ARs are expressed abundantly in cardiomyocytes and the adrenergic signaling cascade is responsible for the control of heart rate (33). Therefore we hypothesized that the change in heart rate observed in anesthetized rats and isolated perfused rat hearts could be due to interaction of β-cardiotoxin with β-ARs, and hence we performed radioligand binding assays. At first, the non-specific binding of radioligand (−)-[³H]CGP-12177 to the receptor preparations was defined using 2 μM (S)-(−)-propranolol hydrochloride. The radioligand showed only 2-3% non-specific binding to β-AR preparation and 1-2% non-specific binding to β₂-AR preparation. (S)-(−)-propranolol hydrochloride was also used as a positive control in the study and IC₅₀ values for the displacement of bound radioligand to β₁-AR and β₂-AR were determined as 3.5 nM and 0.5 nM, respectively (FIG. 10). The binding affinity (K₁) of (S)-(−)-propranolol hydrochloride to β₁-AR and β₂-AR were calculated as 1.9 nM and 0.2 nM, respectively. These values are in good agreement with the Kᵢ values given by the manufacturer (2.6 nM and 0.2 nM for β₁-AR and β₂-AR, respectively). β-Cardiotoxin showed a dose-dependent displacement of the radioligand (−)-[³H]CGP-12177. IC₅₀ values for the inhibition of ligand binding to β₁-AR and β₂-AR were determined as 10 μM and 5 μM, respectively (FIG. 10, A and B). The Kᵢ for β-cardiotoxin binding to β₁ and β₂ ARs were calculated as 5.3 μM and 2.3 μM, respectively. Thus, β-cardiotoxin induces negative chronotropic effect on the heart rate by binding to β₁-AR in cardiomyocytes. Its interaction with β₂-AR in the bronchi may be responsible for the observed breathing difficulties in mice. It is the first exogenous protein which interacts with β-ARs and hence it was named as β-cardiotoxin.

### Discussion

Cardiovascular diseases (CVD) are widespread and are a major health issue in the developed nations. For example, a recent study revealed that about 71.3 million American adults suffer from one or more forms of CVD (34). CVD causes nearly 1 in every 3 deaths and was the number one killer in the USA as it was the cause for 37.3% deaths in 2003. The economic impact of CVD is huge, and it is estimated that the combined direct and indirect costs of treating CVDs in 2006 would be $403.1 billion (34). Betablockers, which are antagonists of β-ARs, are the drugs of choice in the treatment of CVDs. Most patients (93%) with myocardial infarction are given beta-blockers on arrival at hospitals. They are also prescribed after discharge as a chronic therapy (34). In addition, beta-blockers are used in the treatment of many pathological conditions afflicting the heart and vasculature, such as ventricular arrhythmias, heart failure, digitalis intoxication and fetal tachycardia. Owing to the presence of β-ARs in various non-cardiac tissues, beta-blockers have also been used to treat conditions like, migraine, essential tremor, situational anxiety, alcohol withdrawal, hyperparathyroidism, glaucoma, portal hypertension and gastrointestinal bleeding (35). Most of the beta-blockers in clinical use currently are small molecules belonging to the aryloxypropanolamine class (36). Although they are widely used, physicians have encountered several problems associated with the currently available beta-blockers. All betablockers are available as racemic mixtures of L- and D-enantiomers, where only the L-enantiomer exerts beta-blockade while the D-enantiomer, apart from being inert, may have adverse side effects. For example, in a clinical trial using the D-enantiomer of the widely used beta-blocker sotalol, it was reported that the mortality increased by 65% compared to placebo (37). Many beta-blockers exhibit varying levels of lipophilicity and so have the ability to cross membrane barriers and reach the central nervous system, causing adverse effects like hallucinations and insomnia (36). Some beta-blockers are shown to increase insulin resistance and raise the risk of diabetes (38). Thus, new beta-blockers with high specificity and low side effects are being sought.

Isolation of a Novel Protein from O. hannah Venom

On screening the cDNA library from the venom gland tissue of O. hannah, we identified five new 3FTXs. One of these 3FTXs showed only about 55% sequence identity with conventional CTXs isolated from Naja sp. (FIG. 2B). Subsequently two other reports (39, 40) have described the sequencing of the same toxin and also some closely related isoforms from cDNA libraries (FIG. 2A), which shows the usefulness of this approach for identification of novel low abundant proteins that have eluded detection by conventional approaches. Here we have described a two-step chromatographic approach for the isolation of this novel protein (FIG. 4).

Identification of Unique Secondary Structural Conformation and Folding Intermediate of β-cardiotoxin

CTXs from different cobra venoms have been classified into two distinct structural subclasses based on their CD spectra (41). The secondary structural conformation of β-cardiotoxin is unique compared to both group 1 and group 2 CTXs (naniproin and CM18, respectively) (FIG. 5A) and hence it is structurally different from both classes of CTXs.

Conventional CTXs have a few well conserved residues apart from the eight Cys residues, which are thought to play an important role in maintaining structural integrity of the three-finger fold. Among them are Tyr 22 and Tyr 51, which have been shown by chemical modification to play vital structural and functional (especially Tyr 22) roles in CTXs (42). Replacement of Tyr 22 led to changes in interaction of β-sheet regions between loops I and II and replacement of Tyr 51 led to structural perturbation in the globular core region of the molecule, the overall effect being a destabilized structural core and highly perturbed dynamics in the 3-stranded β-sheet region (43). Unlike conventional CTXs, in β-cardiotoxin there are Val residues in both positions 23 and 53 (homologous to positions 22 and 51 of CTXs) (FIG. 2B). To determine the consequences of these changes on the structure and stability of β-cardiotoxin, we performed thermal denaturation studies and monitored the temperature-induced secondary structural changes using far-UV CD spectroscopy. There are changes in the pattern and content of secondary structure starting from 75° C. in β-cardiotoxin (FIG. 5C), whereas the conventional CTX CM18 is stable up to 85° C. and starts showing signs of unfolding only at 95° C. (FIG. 5B). Unlike CM18 which showed a general reduction in the β-sheet content upon heating, β-cardiotoxin underwent a complete structural transition at 85° C., as new minima appeared at 222-225 nm and 203 nm indicating the presence of α-helical elements (FIG. 5C). To determine the implication of this observation on the overall fold and tertiary structure of the protein, we monitored the near-UV CD spectra at various temperatures (FIG. 6). There was a complete loss of tertiary structure coinciding closely with the structural transition occurring at 85° C. (FIG. 5C). This indicates the formation of a stable folding intermediate with intact secondary structural elements but loss of tertiary structure, which resembles the ‘molten globule’ state (30-32).

The molten globule state has been reported for many different proteins since it was first defined. Essentially, all molten globules possess three distinctive features, namely, compactness, presence of a secondary structure and a lack of rigid tertiary structure. This state has been achieved in different proteins under varied conditions like acidification, moderate concentrations of strong denaturants like urea or guanidine HCl, low concentrations of alcohol or fluoroalcohol (e.g. trifluoroethanol (TFE)), inorganic salt denaturants like sodium perchlorate, acids such as trichloroacetic acid, removal of tightly bound ligand, chemical modifications or site directed mutagenesis and in certain cases high pressure as well as temperature (44). Molten globule states of β-sheet-rich proteins are less studied and have been reported in a few cases like ubiquitin (45), rat fatty acid binding protein (46), intertleukin-1α (47), CTX analog III (48), α-lactoglobulin (βα-LG) (49), tumor necrosis factor-α (TNF-α) (50) and a few other molecules (44). Only α-LG and TNF-α, among the above proteins, exhibited a structural transition to a-helix associated with the formation of molten globule. The structural transition in α-LG was induced by addition of TFE, which is a known α-helix inducer (49). In the case of murine TNF-α, the structural transition occurred with the increase in temperature but without the addition of any chemical agent. It was postulated that once the non-local interactions leading to β-sheet formation were disrupted due to high temperatures, the local interactions were established resulting in the formation of α-helices due to a high α-helix forming propensity of murine TNF-α(50). The molten globule state of CTX analog III was achieved using high temperatures as well as addition of chemical denaturants (48). However, no structural transitions were observed at higher temperatures (48).

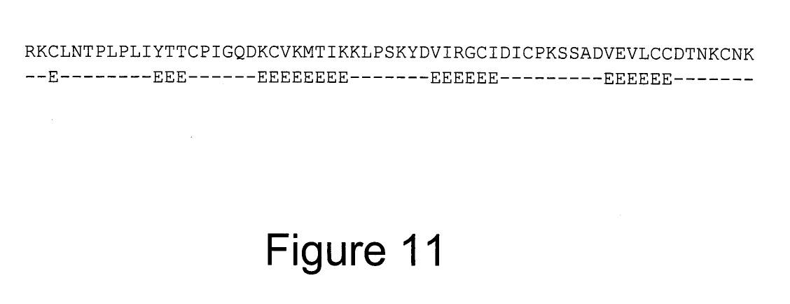

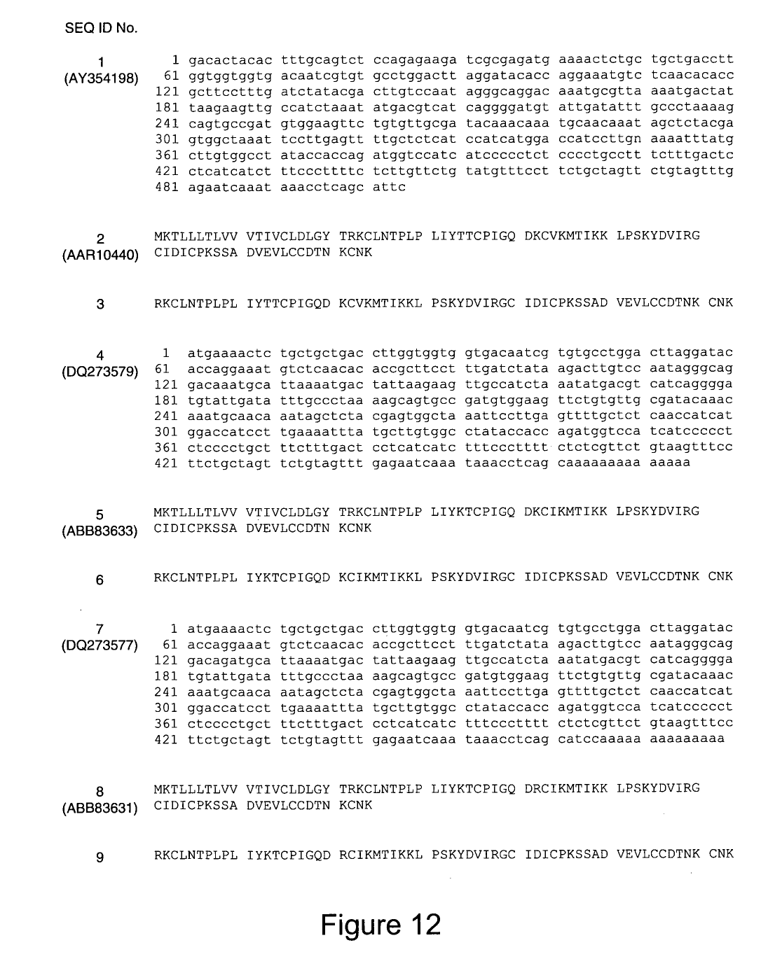

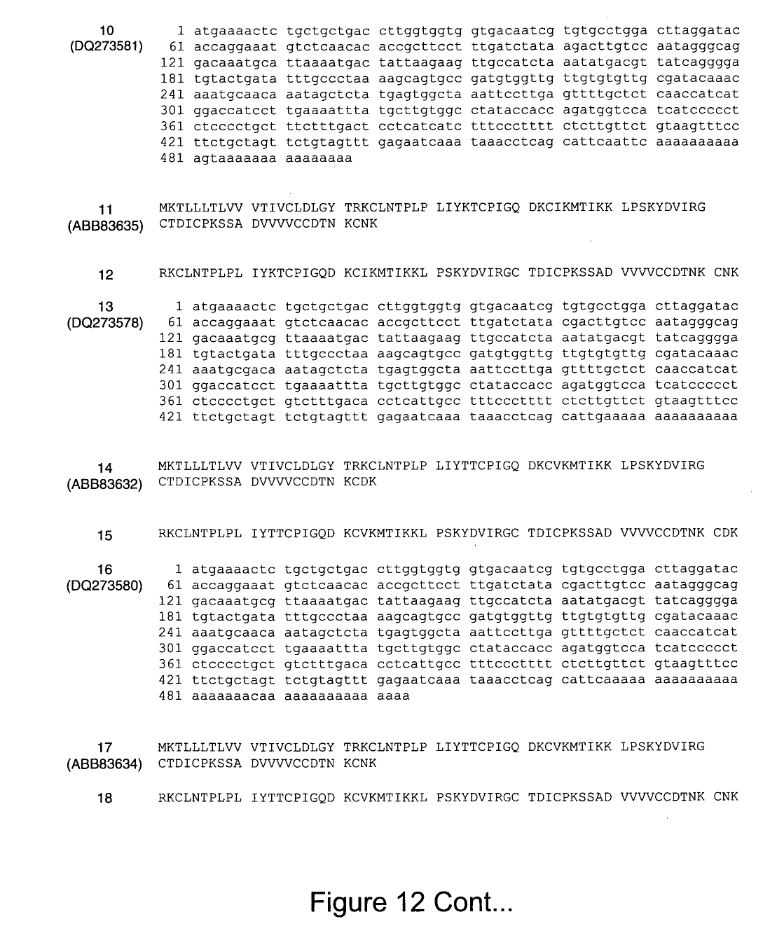

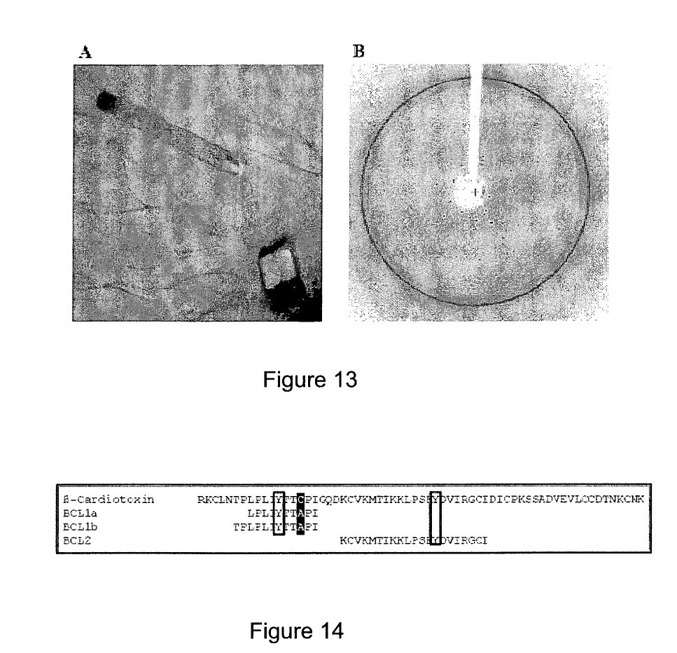

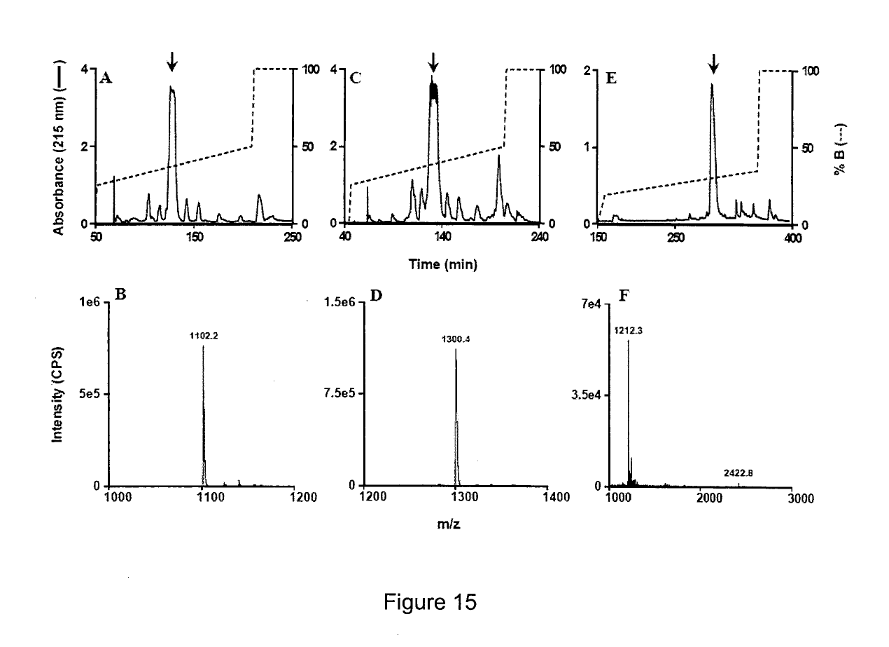

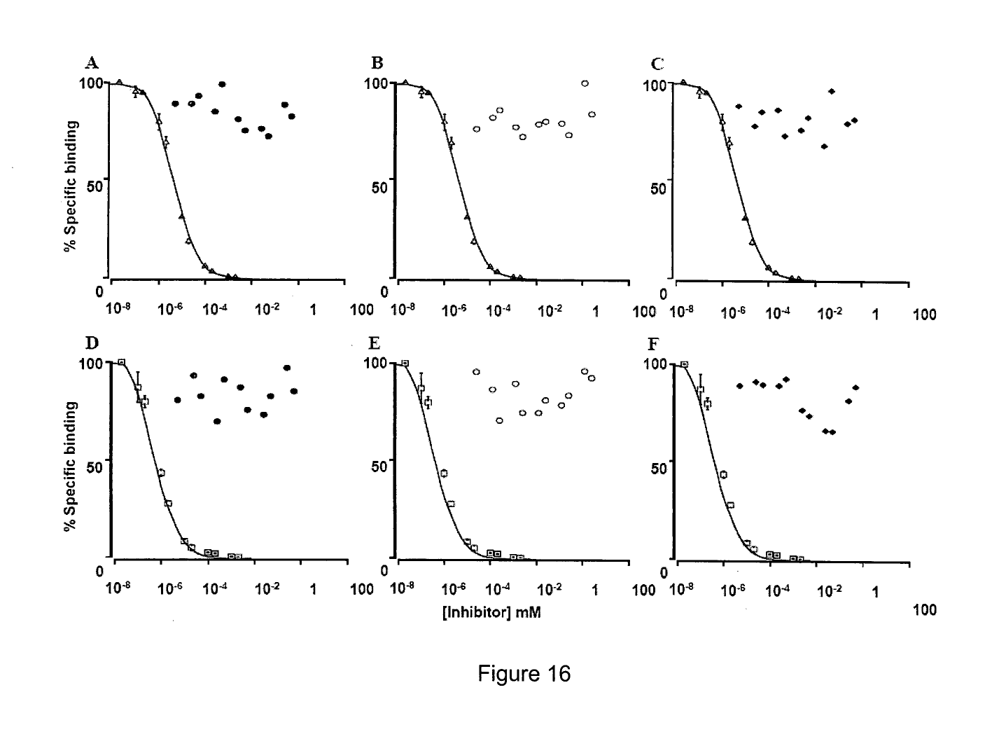

As shown here, β-cardiotoxin has a stable folding intermediate with α-helical elements at 85° C. (FIG. 5C). Interestingly, unlike TNF-α, it has no α-helix forming propensity as calculated according to Pollastri and McLysaght (51) (FIG. 11). Nonetheless it undergoes a structural transition (from β-sheet to α-helix) at higher temperatures without the addition of any external agent or a reduction in pH. At lower temperatures it switches from α-helix to β-sheet (FIG. 5C, inset). Thus β-cardiotoxin could be used as a tool to study the non-hierarchical model of protein folding (52).

Particularly, it may be a useful tool to understand the mechanism involved in α-helix to β-sheet structural transition as the unfolding process is completely reversible (FIG. 5C, inset). Such α-helix to β-sheet structural transition plays a crucial role in neurodegenerative diseases (44). A better understanding of such natural events may be useful in the design and development of agents that would block such structural transitions.

### β-Cardiotoxin Belongs to a New Class of 3FTXs

3FTXs constitute about 50% of the weight of most elapid and hydrophid venoms, and are the leading cause of death and morbidity as they are highly lethal (53). As mentioned in the introduction, despite the similarity in overall protein fold, they target different receptors, ion channels or proteins to exhibit pharmacological effects. As shown here, β-cardiotoxin exhibits unique biological effects compared to any of the 3FTXs known. Although its amino acid sequence shows similarity to CTXs (approximately 55% identity), it is structurally and functionally distinct from conventional CTXs. β-Cardiotoxin was non-lethal up to a dose of 10 mg/kg, in contrast to CTXs, which are highly lethal proteins with LD₅₀ values in the range of 1 to 2 mg/kg (1). Unlike CTXs, which show potent hemolytic activity (54-56), β-cardiotoxin failed to show hemolytic activity on washed human erythrocytes (FIG. 7B). Further, unlike CTXs that cause an increase in heart rate when injected into anesthetized rats (FIGS. 8, B and D) (57), β-cardiotoxin caused a dose-dependent decrease in heart rate indicated by the prolongation of successive QRS complexes in the ECG recording (FIGS. 8, C-E). We also found that it can act directly on the cardiac tissue causing a marked reduction in heart rate in Langendorff preparations of perfused rat hearts without affecting the contractility (FIGS. 9, B-D). β-Cardiotoxin binds directly to β₁- and β₂-ARs. The Kᵢ values indicate that it has higher affinity for β₂-AR compared to β₁-AR (5.3 μM and 2.3 μM for β₁- and β₂-ARs, respectively). This is the first report of an exogenous peptide targeting β-ARs. Thus it is a new class of 3FTXs. The proteins shown in FIG. 2A are highly similar to β-cardiotoxin (92-96% identity) and the substitutions are mostly in the core region and not the extended loops (FIG. 3B). Therefore, we hypothesize that these proteins are likely to be functionally related to β-cardiotoxin. Thus these 3FTXs, although identified as CTXs and short-chain neurotoxins, may probably act as betablockers. The structure-function relationship studies of these proteins will indicate whether the few amino acid changes disrupt their interaction with β₁- and β₂-ARs.

Adrenergic receptors (ARs) are G protein-coupled receptors (GPCRs) that are expressed in a wide variety of tissues and are important functional regulators of various systems like cardiac, pulmonary, vascular, endocrine and central nervous systems (58). ARs in the heart act as a link between sympathetic nervous system and cardiovascular system and play an important role in the rapid regulation of myocardial function. β₁-AR is the predominant receptor subtype expressed in cardiomyocytes (33). The proportion of β₁- and β₂-ARs present in heart varies with species, age and developmental stage (33). β₃-ARs are less abundant in the heart and their functional role is poorly understood (59). In vivo and in vitro assays show that β₁-ARs play a predominant role in modulating the heart rate and the force of contraction in murine hearts (59). On the other hand, under normal conditions β₂-ARs are expressed abundantly in bronchi of lungs and regulate its contractility (36). Thus specificity of beta-blockers is critical in the treatment of patients with CVDs, particularly those with breathing disorders. As currently available beta-blockers have some crossreactivity with β₂-AR, they are not suitable for this subset of CVD patients. Recent studies have shown that β₂-ARs are the only subtype of β-ARs to be expressed on the membranes of major cell types in the skin like keratinocytes (60), fibroblasts (61) and melanocytes (62). Pullar and co-workers demonstrated that β₂-AR activation leads to delay in skin wound healing (63). They went on to exploit this finding and showed that β₂-AR blockade using non-specific beta-blockers accelerated wound healing process (58). Hence, this could be another application for therapeutic use of β-cardiotoxin.

The interaction of β-cardiotoxin with β₁-AR leads to decrease in heart rate, whereas its interaction with β₂-AR leads to the breathing difficulties observed in mice. Thus β-cardiotoxin represents a new class of protein beta-blockers that can be exploited as a novel therapeutic prototype. The structure-function studies and suitable engineering may help in the development of new classes of either β₁-AR-specific beta-blockers (for systemic administration) that would help in the treatment of CVDs or β₂-AR-specific beta-blockers (for topical administration) that would help in wound healing.

In summary, we have described the identification and isolation of β-cardiotoxin which is the first member of a new class of 3FTXs. Although closely related to conventional CTXs, it has unique structural and functional features. We have characterized a unique ‘molten globule’ intermediate in the thermal unfolding process of β-cardiotoxin. Functionally it directly acts on cardiac tissue causing bradycardia. These effects are mediated through its interaction with β-ARs. These proteins could serve as prototypes for rational design of highly specific and effective beta-blocking peptides having reduced side effects. Thus this is the first exogenous protein beta-blocker.

### Example 2

### Introduction

Snake venoms have provided a number of novel ligands with therapeutic potential. We have described the identification and isolation of β-cardiotoxin, which is the first member of a new class of three-finger toxins (3FTXs). Although it shows sequence homology to conventional cardiotoxins (CTXs), it has unique structural and functional features. Conventional CTXs are highly lethal proteins with LD₅₀ values in the range of 1 to 2 mg/kg (Hider et al., 1991). They show potent hemolytic activity (Osorio e Castro et al., 1989, Louw and Visser, 1978 and Hider and Khader, 1982) and cause an increase in heart rate (tachycardia) when injected into anesthetized rats (Sun and Walker, 1986). In contrast, β-cardiotoxin is nonlethal up to a dose of 10 mg/kg, does not show haemolytic activity on washed human erythrocytes and most importantly causes a dose-dependent decrease in heart rate (bradycardia) with a prolongation of successive QRS complexes in the ECG recordings (Rajagopalan et al., 2007). We also found that it acts directly on the cardiac tissue causing a marked reduction in heart rate (negative chronotropism) in Langendorff preparations of perfused rat hearts without affecting the contractility (inotropism). Further, we have shown that the above mentioned pharmacological effects are due to its direct binding to β1- and β2-adrenergic receptors (ARs) (Rajagopalan et al., 2007). Thus, β-cardiotoxin is the first exogenous protein beta-blocker. Therefore, it is important to understand the structure and structure-function relationships of this unique class of beta-blocker. These studies may help in the rational design of highly specific and effective beta blocking peptides.

All known beta blockers belong to a single small molecule family called aryloxypropanolamine. Structure-function relationship studies of these molecules revealed the importance of the aromatic ring structure for its interaction with β-adrenergic receptors (β-ARs) (Kobilka, 2007 and Rosenbaum et al., 2007). Therefore, we hypothesised that one of the Tyr residues found in the β-cardiotoxin may be involved in its interaction with β-ARs. Thus we designed three peptides from β-cardiotoxin. While designing these three peptides we have also incorporated design ideas based on the ‘proline-bracket hypothesis’ (Kini and Evans, 1994). These peptides were synthesized by solid-phase peptide synthesis and purified by reverse phase-HPLC (RP-HPLC). Their interaction with β-ARs was determined by competitive binding assays using a radiolabled ligand. We have also initiated studies on the determination of its three-dimensional structure by X-ray crystallography.

### Experimental Procedures

### Materials

Standard Fmoc-L-amino acid hydroxyl derivatives, Fmoc-L-Ile-PEG-PS (polyethylene glycol-polystyrene) support resin, N,N-dimethylformamide (DMF), trifluoroacetic acid (TFA), 20% piperidine in DMF, O-(7-azabenzotriazol-1-yl)-1,1,3,-3-tetramethyluronium hexafluorophosphate (HATU), and N,N-diisopropylethylamine (DIPEA) were purchased from Applied Biosystems Asia Pte Ltd (Foster City, Calif.). Jupiter C₁₈ (5μ, 300 Å, 10×250 mm) were purchased from Phenomenex (Torrance, Calif., USA). 1,2-Ethanedithiol and thioanisole were obtained from Fluka/Riedel-de Haen (Sigma Aldrich, St. Louis, Mo.). All other chemicals and reagents used were of analytical grade.

### Crystallization

β-Cardiotoxin was purified by a two-step chromatography approach as described previously (Rajagopalan et al., 2007). Crystallization trials for β-cardiotoxin were conducted using the hanging drop vapor-diffusion method and a wide range of conditions were tested using Hampton Research Crystal Screens I, II and QIAgen PACT suite. The protein concentration was kept at 15 mg/ml and the drops were prepared by mixing equal volumes (1 μl) of protein solution (dissolved in 25 mM Tris buffer pH 7.4) and crystallization solution. The screens were set up at 295 K using VDX plates from Hampton Research. 500 μl reservoir solution was placed in each well. The initial screen identified a PEG-based crystallization condition. By systematic optimization around the preliminary condition, we obtained diffraction-quality crystals of β-cardiotoxin. The best crystals were from a condition consisting of 0.1 M SPG buffer pH 7, 25% PEG 1500 with an additive of 0.1 μl of 0.1 M spermidine.

### Data Collection

We have collected a complete native data set for β-cardiotoxin. The crystals were picked up with a nylon cryo-loop and frozen at 100 K in a nitrogen-gas cold stream (Cryostream cooler; Oxford Cryosystems, Oxford, England). Data was collected at the in house Rigaku Xray generator. Data sets were processed using HKL-2000 (Otwinowski & Minor, 1997). The crystals diffract to 3.2 Å resolution. The space group was P2, with unit cell parameters a=54.64, b=90.90, c=88.82 Å. The Matthews coefficient VM of 3.10 Å 3/Da (Matthews, 1968) is within the expected range for 10 monomers in the asymmetric unit and corresponds to a solvent content of 60.33%.

### Peptide Synthesis and Purification

Three peptides (BCL1a, BCL1b and BCL 2) were synthesized using solid-phase peptide synthesis methods on an Applied Biosystems Pioneer Model 433A Peptide Synthesizer. Fmoc groups of amino acids were removed by 20% v/v piperidine in N,N-dimethylformamide and coupled using HATU/DIPEA in situ neutralization chemistry. All peptides were synthesized on preloaded polyethylene glycol polystyrene (PEG-PS) resins.

Cleavage by a mixture of trifluoroacetic acid/1,2-ethanedithiol/thioanisole/water released peptide acids (—COOH). Synthetic peptides were purified by reverse phase-HPLC on an AKTA purifier (GE Healthcare, Uppsala, Sweden) with a Jupiter C₁₈ (5μ, 300 Å, 10 mm×250 mm) column that was equilibrated with 0.1% trifluoroacetic acid and the proteins eluted with a linear gradient of 80% acetonitrile in 0.1% trifluoroacetic acid. The fractions were collected and directly injected into an API-300 liquid chromatography/tandem mass spectrometry system (PerkinElmer Life Sciences, Wellesley, Mass., USA) for mass determination. Fractions showing the expected molecular mass were pooled and lyophilized. Air oxidation was carried out for BCL2 to form the disulfide linkage. Oxidation reactions were quenched by adding glacial acetic acid to adjust pH to 4.0 before separation by RP-HPLC.

### Competitive Binding Assay

Cloned human β-adrenergic receptor subtypes 1 and 2 (β1 and (β2) produced in Sf9 cells were purchased from PerkinElmer Life and Analytical Sciences (Boston, Mass., USA). The radioligand (−)-[³H]CGP-12177 was purchased from GE Healthcare (Buckinghamshire, UK). The competitive binding assay was carried out using the method described by Sharpe et al., and a few recommendations by the receptor manufacturers (PerkinElmer Life and Analytical Sciences) were also incorporated. For the competitive binding studies, we used 0.15 nM (−)-[³H]CGP-12177, β1 or β2 and β-cardiotoxin ranging from 5 nM to 10 μM in 75 mM Tris-HCl buffer containing 12.5 mM MgCl2 and 2 mM EDTA, pH 7.4. Total reaction volume was 1050 μl. Nonspecific binding was determined to be 2-3% for β1 and 1-2% for β2 by the inclusion of 2 μM (S)-(−)-propranolol hydrochloride. After 60 min incubation at room temperature, the reaction mixtures were filtered through Whatman GF/C glass microfibre filters (Maidstone, England) presoaked in ice-cold wash buffer (50 mM Tris-HCl buffer, pH 7.4). The filters were washed nine times with 500 μl (each time) of ice-cold wash buffer. A Beckman LS3801 liquid scintillation counter was used to measure the radioactivity retained on the washed filters. The binding affinity Kᵢ was calculated from the IC₅₀ using the equation of Cheng and Prusoff,

Kᵢ=IC₅₀+{1+([Radioligand]/K_{d})}

where, the IC₅₀ (concentration of the inhibitor that displaces 50% of bound ligand) values were determined by plotting the percent specific binding in the y-axis vs. log [molar concentration of protein used] in the x-axis, K_{d} is the binding affinity of the radioligand to the receptor.

### Results and Discussion

Crystallization of β-cardiotoxin

β-Cardiotoxin is a novel member of the 3FTX family. 3FTXs are the most abundant and well-characterized non-enzymatic family of snake venom toxins found mostly in hydrophid (sea snakes) and elapid (cobras and kraits) venoms. All members of this family share a similar fold consisting of three finger-like loops made of β-sheet structure, projecting from a globular core and stabilized by four or five intramolecular disulfide linkages giving rise to the compact “β-cross” motif (Endo and Tamiya, 1987, Kini 2002, Nirthanan et al., 2003, Tsetlin 1999, and Harrison and Sternberg, 1996). Though the members of this large family of proteins share similar structural scaffold, it has been shown that there are subtle differences that could contribute to the difference in affinity and specificity (Rees et al., 1987 and Bilwes et al., 1994). Thus it is very essential to solve the 3-D structure of this novel protein to facilitate in the rational design of bioactive peptides.

We have successfully identified the crystallization condition for the protein after screening 192 conditions. β-Cardiotoxin was crystallized by the hanging-drop vapour-diffusion method by equilibration against 0.1 M SPG buffer pH 7 containing 0.1 μl of 0.1M spermidine and 25% PEG 1500 as the precipitating agent (FIG. 13A). The crystals belong to space group P2, with unit cell parameters a=54.64, b=90.90, c=88.82 Å. There are 10 monomers in the asymmetric unit. The crystals diffract to 3.2 Å resolution (FIG. 13B). We are currently trying to optimize the cryo conditions to obtain higher resolution data with lower mosaicity.

### Structure-Function Relationship Studies

β-Cardiotoxin is the first exogenous protein reported to bind to and inhibit the β-ARs. All known beta-blockers belong to a single small molecule family called aryloxypropanolamine. Although they are widely used, physicians have encountered several problems associated with the currently available beta-blockers. All beta-blockers are available as racemic mixtures of L and D-enantiomers, where only the L-enantiomer exerts beta-blockade while the D-enantiomer, apart from being inert, may have adverse side effects. For example, in a clinical trial using the D-enantiomer of the widely used beta-blocker sotalol, it was reported that the mortality increased by 65% compared to placebo. Many beta-blockers exhibit varying levels of lipophilicity and so have the ability to cross membrane barriers and reach the central nervous system, causing adverse effects like hallucinations and insomnia. Some beta-blockers are shown to increase insulin resistance and raise the risk of diabetes (Griffith, 2003, Waldo et al., 1996 and Gress et al., 2000). Another major issue with beta blockers is that many beta blockers like esmolol exert a direct inhibitory effect on membrane Ca²⁺ channels apart from acting as beta-blocking agents. This inhibition of membrane currents would lead to pronounced negative inotropism leading to adverse complications like severe reduction of blood pressure (Arlock et al., 2005). However, such a problem was not encountered when β-cardiotoxin was injected into animals. β-Cardiotoxin induced a negative chronotropism but did not affect the inotropism of animal hearts (Rajagopalan et al., 2007).

All the natural ligands of β-ARs have an aromatic ring structure. This moiety has been shown to be very important for interaction with trans-membrane helices TM 3, TM 5 and TM 6 (Kobilka, 2007). There are only two aromatic amino acid residues in the β-cardiotoxin sequence, Tyr 12 and Tyr 34 (FIG. 14). Thus, we hypothesize that these Tyr residues may play an important role in the interaction of β-cardiotoxin with β-ARs. To test this hypothesis we designed three short peptides from the β-cardiotoxin sequence (FIG. 14). We used design ideas from the “Proline-bracket” hypothesis proposed by Kini and Evans for the design of BCL1a (SEQ ID NO.46 and BCL1b (SEQ ID No.47). BCL2 (SEQ ID No.48) was designed as a circular peptide by placing one Cys on either end of the peptide (FIG. 14). The designed peptides were synthesized by solid-phase chemical synthesis method and purified by RP-HPLC (FIGS. 15A, C and E). The masses of the purified peptides were determined by ESI-MS and we found that they matched exactly with the calculated molecular weights (FIGS. 15B, D and F). BCL2 was circularized after synthesis by oxidation of the free Cys residues. We performed competitive binding assay using the radioligand (−)-[³H]CGP-12177 to determine the interaction of the three peptides with cloned human β-AR subtypes 1 and 2 (FIG. 16). These peptides were designed without any structural inputs as the 3-D structure of β-cardiotoxin is not yet solved.

### CONCLUSIONS

In summary, we have identified the crystallization condition for obtaining good quality crystals of β-cardiotoxin. We have also designed three peptides based on published information about β-ARs and also the “Proline-bracket” hypothesis.

### REFERENCES

1. Harvey, A. L. (1991) Snake Toxins pp. 1-34 Pergamon Press, New York
2. Lee, C. Y. (1979) Snake Venoms pp. 159-212 Springer-Verlag, N.Y.
3. Higuchi, S., Murayama, N., Saguchi, K., Ohi, H., Fujita, Y., Camargo, A. C., Ogawa, T., Deshimaru, M., and Ohno, M. (1999) Immunopharmacology 44, 129-135
4. O'Shea, J. C., and Tcheng, J. E. (2002) Expert Opin. Pharmacother. 3, 1199-1210
5. Marcinkiewicz, C. (2005) Curr. Pharm. Des. 11, 815-827
6. Huang, F., and Hong, E. (2004) Curr. Med. Chem. Cardiovasc. Hematol. Agents 2, 187-196
7. Plosker, G. L., and Ibbotson, T. (2003) PharmacoEconomics 21, 885-912
8. Kondo, K., and Umemura, K. (2002) Clin. Pharmacokinet. 41, 187-195
9. McClellan, K. J., and Goa, K. L. (1998) Drugs 56, 1067-1080
10. Sherman, D. G. (2002) Curr. Med. Res. Opin. 18, Suppl. 2, s48-s52
11. Chang, C. C. (1999) J. Biomed. Sci. 6, 368-375
12. Endo, T., and Tamiya, N. (1987) Pharmacol. Ther. 34, 403-451
13. Kini, R. M. (2002) Clin. Exp. Pharmacol. Physiol. 29, 815-822
14. Nirthanan, S., Gopalakrishnakone, P., Gwee, M. C., Khoo, H. E., and Kini, R. M. (2003) Toxicon 41, 397-407
15. Tsetlin, V. (1999) Eur. J. Biochem. 264, 281-286
16. Harrison, P. M., and Sternberg, M. J. E. (1996) J. Mol. Biol. 264, 603-623
17. Potter, L. T. (2001) Life Sci. 68, 2541-2547
18. Karlsson, E., Mbugua, P. M., and Rodriguez-Ithurralde, D. (1984) J. Physiol (Paris) 79, 232-240
19. Yasuda, O., Morimoto, S., Jiang, B., Kuroda, H., Kimura, T., Sakakibara, S., Fukuo, K., Chen, S., Tamatani, M., and Ogihara, T. (1994) Artery 21, 287-302
20. McDowell, R. S., Dennis, M. S., Louie, A., Shuster, M., Mulkerrin, M. G., and Lazarus, R. A. (1992) Biochemistry 31, 4766-4772
21. Kumar, T. K. S., Jayaraman, G., Lee, C. S., Arunkumar, A. I., Sivaraman, T., Samuel, D., and Yu, C. (1997) J. Biomol. Struc. Dyn. 15, 431-463
22. Banerjee, Y., Mizuguchi, J., Iwanaga, S., and Kini, R. M. (2005) J. Biol. Chem. 280, 42601-42611
23. Wu, P. L., Lee, S. C., Chuang, C. C., Mori. S., Akakura, N., Wu, W. G., and Takada, Y. (2006) J. Biol. Chem. 281, 7937-7945)
24. Fry, B. G., Wuster, W., Kini, R. M., Brusic, V., Khan, A., Venkataraman, D., and Rooney, A. P. (2003) J. Mol. Evol. 57, 110-129
25. Quick, A. J. (1935) J. Biol. Chem. 109, 73-74
26. Langdell, R. D., Wagner, R. H., and Brinkhous, K. M. (1953) J. Lab. Clin. Med. 41, 637-647
27. Cheng, Y., Prusoff, W. H. (1973) Biochem. Pharmacol. 22, 3099-3108
28. Pung, Y. F., Wong, P. T., Kumar, P. P., Hodgson, W. C. and Kini, R. M. (2005) J. Biol. Chem. 280, 13137-13147
29. Schmid, F. (2005) Protein Folding Handbook 1, pp. 22-44 Wiley-VCH, Germany
30. Kuwajima, K., Nitta, K., Yoneyama, M., and Sugai, S. (1976) J. Mol. Biol. 106, 359-373
31. Dolgikh, D. A., Gilmanshin, R. I., Brazhnikov, E. V., Bychkova, V. E., Semisotnov, G. V., Venyaminov, S. Yu., and Ptitsyn, O. B. (1981) FEBS Lett. 136, 311-315
32. Ohgushi, M., and Wada, A. (1983) FEBS Lett. 164, 21-24
33. Xiang, Y., and Kobilka, B. K. (2003) Science 300, 1530-1532
34. Thom, T., Haase, N., Rosamond, W., Howard, V. J., Rumsfeld, J., Manolio, T., Zheng, Z. J., Flegal, K., O'Donnell, C., Kittner, S., Lloyd-Jones, D., Goff, D. C., Hong, Y., Adams, R., Friday, G., Furie, K., Gorelick, P., Kissela, B., Marler, J., Meigs, J., Roger, V., Sidney, S., Sorlie, P., Steinberger, J., Wasserthiel-Smoller, S., Wilson, M., and Wolf, P. (2006) Circulation 113, 85-151
35. Hollenberg, N. K. (2005) Am. J. Hypertens. 18, 165s-168s
36. Griffith, R. K. (2003) Burger's Medicinal Chemistry and Drug Discovery 6, pp. 1-37 John Wiley and Sons, Inc.
37. Waldo, A. L., Camm, A. J., deRuyter, H., Friedman, P. L., MacNeil, D. J., Pauls, J. F., Pitt, B., Pratt, C. M., Schwartz, P. J., Veltri, E. P. (1996) Lancet 348, 7-12
38. Gress, T. W., Nieto, F. J., Shahar, E., Wofford, M. R., and Brancati, F. L. (2000) N. Engl. J. Med. 342, 905-912
39. He, Y. Y., Lee, W. H., and Zhang, Y. (2004) Toxicon 44,295-303
40. Li, J., Zhang, H., Liu, J., and Xu, K. (2006) Biochem J. 398, 233-242
41. Grognet, J. M., Menez, A., Drake, A., Hyashi, K., Morrison, I. E., and Hider, R. C. (1988) Eur. J. Biochem. 172, 383-388
42. Gatineau, E., Toma, F., Montenay-Garestier, T., Takechi, M., Fromageot, P., and Menez, A. (1987) Biochemistry 26, 8046-8055
43. Roumestand, C., Gilquin, B., Tremeau, O., Gatineau, E., Mouawad, L., Menez, A., and Toma, F. (1994) J. Mol. Biol. 243, 719-735
44. Arai, M., and Kuwajima, K. (2000) Adv. Protein Chem. 53, 209-282
45. Briggs, M. S., and Roder, H. (1992) Proc. Natl. Acad. Sci. U.S.A. 89, 2017-2021
46. Ropson, I. J., and Frieden, C. (1992) Proc. Natl. Acad. Sci. U.S.A. 89, 7222-7226
47. Varley, P., Gronenborn, A. M., Christenson, H., Wingfield, R. T., Pain, R. H., and Clore, G. M. (1993) Science 260, 1110-1111
48. Kumar, T. K. S., Jayaraman, G., Lee, C. S., Sivaraman, T., Lin, W. Y., and Yu, C. (1995) Biochem. Biophys. Res. Commun. 207, 536-543
49. Shiraki, K., Nishikawa, K., and Goto, Y. (1995) J. Mol. Biol. 245, 180-194
50. Narhi, L. 0., Philo, J. S., Li, T., Zhang, M., Samal, B., and Arakawa, T. (1996) Biochemistry 35, 11447-11453
51. Pollastri, G., and McLysaght, A. (2005) Bioinformatics 21, 1719-1720
52. Lim, V. I. (1978) FEBS Lett. 89, 10-14
53. Boffa, M. C., Barbier, D., and deAngulo, M. (1983) Thrombosis Research 32, 635-640
54. Osorio e Castro, V. R., and Vernon, L. P. (1989) Toxicon 27, 511-517
55. Louw, A. I., and Visser, L. (1978) Biochim. Biophys. Acta 512, 163-171
56. Hider, R. C., and Khader, F. (1982) Toxicon 20, 175-179
57. Sun, J. J., and Walker, J. A. (1986) Toxicon 24, 233-245
58. Pullar, C. E., Rizzo, A., and Isseroff, R. R. (2006) J. Biol. Chem. 281, 21225-21235
59. Rohrer, D. K., Chruscinski, A., Schauble, E. H., Bernstein, D., and Kobilka, B. K. (1999) J. Biol. Chem. 274, 16701-16708
60. Schallreuter, K. U., Wood, J. M., Pittelkow, M. R., Swanson, N. N., and Steinkraus, V. (1993) Arch. Dermatol. Res. 285, 216-220
61. McSwigan, J. D., Hanson, D. R., Lubiniecki, A., Heston, L. L., and Sheppard, J. R. (1981) Proc. Natl. Acad. Sci. U.S.A. 78, 7670-7673
62. Gillbro, J. M., Marles, L. K., Hibberts, N. A., and Schallreuter, K. U. (2004) J. Invest. Dermatol. 123, 346-353
63. Pullar, C. E., Grahn, J. C., Liu, W., and Isseroff, R. R. (2006) FASEB J. 20, 76-86
64. Arlock, P., Wohlfart, B., Sjoberg, T., and Steen, S. (2005). Scand. Cardiovasc. J. 39, 250-254.
65. Bilwes, A., Rees, B., Moras, D., Menez, R. and Menez, A. (1994). J Mol Biol. 239, 122-36.
66. Cheng, Y., and Prusoff, W. H. (1973). Biochem. Pharmacol. 22, 3099-3108.
67. Hider, R. C., Karlsson, E., and Namiranian, S. (1991) Separation and purification of toxins from snake venoms. In Snake Toxins (Harvey, A. L., ed) pp. 1-34, Pergamon Press, Inc., New York, N.Y., U.S.A.
68. Kini R. M. and Evans H. J. (1994). Current topics in peptide and prot. res. 1, 1994.
69. Kobilka, B. K. (2007). Biochim Biophys Acta. 1768, 794-807.
70. Matthews, B. W. (1968). J. Mol. Biol. 33, 491-497.
71. Osorio e Castro, V. R., and Vernon, L. P. (1989) Hemolytic activity of thionin from Pyrularia pubera nuts and snake venom toxins of Naja naja species: Pyrularia thionin and snake venom cardiotoxin compete for the same membrane site. Toxicon. 27, 511-517.
72. Otwinowski, Z. & Minor, W. (1997). Methods Enzymol. 276, 307-326.
73. Pitt, B., Pratt, C. M., Schwartz, P. J., and Veltri, E. P. (1996). Lancet 348, 7-12.
74. Rajagopalan, N., Pung, Y. F., Zhu, Y. Z., Wong, P. T., Kumar, P. P. and Kini, R. M. (2007). FASEB J. 21, 3685-95.
75. Rees, B., Samama, J. P., Thierry, J. C., Gilibert, M., Fischer, J., Schweitz, H., Lazdunski, M. and Moras, D. (1987). Proc Natl Acad Sci USA. 84, 3132-6.
76. Rosenbaum, D. M., Cherezov, V., Hanson, M. A., Rasmussen, S. G., Thian, F. S., Kobilka, T. S., Choi, H. J., Yao, X. J., Weis, W. I., Stevens, R. C., Kobilka, B. K. (2007). Science. [Epub ahead of print].
77. Sharpe, I. A., Gehrmann, J., Loughnan, M. L., Thomas, L., Adams, D. A., Atkins, A., Palant, E., Craik, D. J., Adams, D. J., Alewood, P. F., and Lewis, R. J. (2001). Nat. Neurosci. 4, 902-907.
78. Sun, J. J., and Walker, J. A. (1986) Actions of cardiotoxins from the southern Chinese cobra (Naja naja atra) on rat cardiac tissue. Toxicon 24, 233-245.
79. Tsetlin, V. (1999). Eur. J. Biochem. 264, 281-286.
80. Waldo, A. L., Camm, A. J., deRuyter, H., Friedman, P. L., MacNeil, D. J., Pauls, J. F.,

1-14. (canceled)

15. A method of reducing the heart rate of an animal comprising the step of administering to the animal a therapeutically effective amount of a polypeptide having the amino acid sequence of SEQ ID NO:3, or an amino acid sequence having at least 60%, 90% or 95% sequence identity to SEQ ID NO:3.

16. The method according to claim 15 wherein said polypeptide has antagonist action at a β₁- and/or β₂- adrenergic receptor.

17. The method according to claim 16 wherein said polypeptide has a Kᵢ of less than 50 μM to the β₁-adrenergic receptor.

18. The method according to claim 16 wherein said polypeptide has a Kᵢ of less than 50 μM to the β₂-adrenergic receptor.

19. The method according to claim 15 wherein said polypeptide is capable of reducing the heart rate in a mammal.

20. The method according to claim 15 wherein the polypeptide comprises an amino acid sequence chosen from one of SEQ ID NO: 2, 3, 5, 6, 8, 9, 11, 12, 14, 15, 17 or 18 or a polypeptide having at least 60%, 90% or 95% amino acid sequence identity to one of SEQ ID NO: 2, 3, 5, 6, 8, 9, 11, 12, 14, 15, 17 or 18.

21. The method according to claim 15 wherein the polypeptide consists of an amino acid sequence chosen from one of SEQ ID NO: 2, 3, 5, 6, 8, 9, 11, 12, 14, 15, 17 or 18 or a polypeptide having at least 60%, 90% or 95% amino acid sequence identity to one of SEQ ID NO: 2, 3, 5, 6, 8, 9, 11, 12, 14, 15, 17 or 18.

22. The method according to claim 15 wherein the polypeptide comprises SEQ ID NO: 19, or a sequence having at least 80%, 90% or 95% sequence identity to SEQ ID NO: 19, wherein each of X₁—X₇ is chosen from: any single amino acid; or a contiguous sequence of 2, 3, 4, 5, 6, 7, 8, 9 or 10 amino acids of any type and in any combination, and wherein each of X₁—X₇ may be the same or different.

23. The method according to claim 22 wherein the polypeptide comprises an amino acid sequence having a plurality of contiguous sequence components each of those sequence components having at least 80% sequence identity with one of SEQ ID NO: 20, 21, 23, 24 and 26 and being:
(i) the same respective length;
(ii) 1 or 2 amino acids longer; or
(iii) 1 or 2 amino acids shorter,
wherein the sequence components are in corresponding linear position in the polypeptide amino acid sequence, as compared with the linear position of SEQ ID NO: 20, 21, 23, 24 and 26 in the polypeptide of SEQ ID NO:19.

24. The method according to claim 22 wherein:
X₁ is a single amino acid chosen from T or K;
X₂ is a single amino acid chosen from K or R;
X₃ is a single amino acid chosen from V or I;
X₄ is a single amino acid chosen from I or T;
X₅ is a single amino acid chosen from E or V;
X₆ is a single amino acid chosen from L or V; and
X₇ is a single amino acid chosen from N or D.

25. A beta-blocker medicament having antagonist activity towards the β₁- and/or β₂-adrenergic receptors, the medicament comprising a polypeptide having the amino acid sequence of SEQ ID NO:3, or an amino acid sequence of at least 60%, 90% or 95% sequence identity to SEQ ID NO:3.

26. The beta-blocker medicament according to claim 25 wherein said polypeptide has a Kᵢ of less than 50 μM to the β₁-adrenergic receptor.

27. The beta-blocker medicament according to claim 25 wherein said polypeptide has a Kᵢ of less than 50 μM to the β₂-adrenergic receptor.

28. The beta-blocker medicament according to claim 25 wherein said polypeptide is capable of reducing the heart rate in a mammal.

29. The beta-blocker medicament according to claim 25 wherein the polypeptide comprises an amino acid sequence chosen from one of SEQ ID NO: 2, 3, 5, 6, 8, 9, 11, 12, 14, 15, 17 or 18 or a polypeptide having at least 60%, 90% or 95% amino acid sequence identity to one of SEQ ID NO: 2, 3, 5, 6, 8, 9, 11, 12, 14, 15, 17 or 18.

30. The beta-blocker medicament according to claim 25 wherein the polypeptide consists of an amino acid sequence chosen from one of SEQ ID NO: 2, 3, 5, 6, 8, 9, 11, 12, 14, 15, 17 or 18 or a polypeptide having at least 60%, 90% or 95% amino acid sequence identity to one of SEQ ID NO: 2, 3, 5, 6, 8, 9, 11, 12, 14, 15, 17 or 18.

31. The beta-blocker medicament according to claim 25 wherein the polypeptides comprises SEQ ID NO:19, or a sequence having at least 80% sequence identity to SEQ ID NO:19, wherein each of X₁—X₇ is chosen from: any single amino acid; or a contiguous sequence of 2, 3, 4, 5, 6, 7, 8, 9 or 10 amino acids of any type and in any combination, and wherein each of X₁—X₇ may be the same or different.

32. The beta-blocker medicament according to claim 30 wherein the polypeptide comprises an amino acid sequence having a plurality of contiguous sequence components each of those sequence components having at least 80% sequence identity with one of SEQ ID NO: 20, 21, 23, 24 and 26 and being:
(i) the same respective length;
(ii) 1 or 2 amino acids longer; or
(iii) 1 or 2 amino acids shorter,
wherein the sequence components are in corresponding linear position in the polypeptide amino acid sequence, as compared with the linear position of SEQ ID NO: 20, 21, 23, 24 and 26 in the polypeptide of SEQ ID NO:19.

33. The beta-blocker medicament according to claim 31 wherein:
X₁ is a single amino acid chosen from T or K;
X₂ is a single amino acid chosen from K or R;
X₃ is a single amino acid chosen from V or I;
X₄ is a single amino acid chosen from I or T;
X₅ is a single amino acid chosen from E or V;
X₆ is a single amino acid chosen from L or V; and
X₇ is a single amino acid chosen from N or D.

34. A medicament comprising a polypeptide having the amino acid sequence of SEQ ID NO:3, or an amino acid sequence of at least 60%, 90% or 95% sequence identity to SEQ ID NO:3, and a pharmaceutically acceptable diluent, adjuvant or carrier.

35. Packaging comprising the medicament of claim 34 and instructions for use of said medicament in a method of reducing the heart rate of an animal.
